(19) United States
(12) Patent Application Publication
(10) Pub. No.: US 2004/0170619 A1
(43) Pub. Date: Sep. 2, 2004

(54) Gene regulation

(76) Inventors: John Girdlestone, Cambridge (UA); Nicole England, United Kingdom (GB); Christopher Demaison, Newington London (UA)

Correspondence Address: ROBINS & PASTERNAK, 1731 EMBARCADERO ROAD, SUITE 230, PALO ALTO, CA, 94303, US

(21) Appl. No.: 10/472,857

(22) PCT Filed: Mar. 19, 2002
(86) PCT No.: PCT/US02/08554

(30) Foreign Application Priority Data
Mar. 19, 2001 (GB) 0106786.7

(51) Int. Cl. A61K 38/47; C12N 9/22; C12N 15/85

(52) U.S. Cl. 424/94.61; 435/199; 435/455

(57) ABSTRACT

We describe a method of regulating expression of a nucleic acid sequence in a primary cell, the method comprising providing a nucleic acid binding polypeptide capable of binding to the nucleic acid sequence, and contacting the nucleic acid binding polypeptide with the nucleic acid sequence in the primary cell to regulate its expression. Nucleic acid binding polypeptides capable of binding to and regulating the expression of a nucleic acid sequence in a primary cell is also disclosed.

### FIELD OF THE INVENTION

This invention relates generally to the field of gene regulation, in particular, regulation of genes in primary cells.

The present invention seeks to solve one or more problems in the prior art.

### SUMMARY OF THE INVENTION

According to a first aspect of the present invention, we provide a method of regulating expression of a nucleic acid sequence in a primary cell, the method comprising providing a nucleic acid binding polypeptide capable of binding to the nucleic acid sequence, and contacting the nucleic acid binding polypeptide with the nucleic acid sequence in the primary cell to regulate its expression.

There is provided, according to a second aspect of the present invention, a nucleic acid binding polypeptide capable of binding to and regulating the expression of a nucleic acid sequence in a primary cell.

Preferably, the nucleic acid sequence comprises an endogenous cellular gene. More preferably, the nucleic acid binding polypeptide is capable of binding to a promoter or other control sequence of the endogenous gene. The nucleic acid binding polypeptide may be provided by expression from an expression vector which is introduced into the primary cell or an ancestor of the primary cell.

In a highly preferred embodiment of the invention, the nucleic acid binding polypeptide comprises a zinc finger polypeptide. The primary cell may comprise an untransformed cell, or alternatively, the primary cell may comprise a tumour or cancer cell.

The nucleic acid binding polypeptide preferably comprises a transcriptional repression domain selected from the group consisting of: a KRAB domain, an engrailed domain and a snag domain. Alternatively, the nucleic acid binding polypeptide comprises a transcriptional activation domain selected from the group consisting of: VP 16, VP64, transactivation domain 1 of the p65 subunit (RelA) of nuclear factor-κB, transactivation domain 2 of the p65 subunit (RelA) of nuclear factor-κB, and the activation domain of CTCF.

Preferably, the primary cell is introduced into an organism. More preferably, the nucleic acid sequence is capable of encoding erythropoietin (EPO) or TNF receptor 1 (TNFR1).

We provide, according to a third aspect of the present invention, a primary cell comprising an exogenous nucleic acid binding polypeptide, the nucleic acid binding polypeptide capable of regulating the expression of a nucleic acid sequence of the primary cell.

As a fourth aspect of the present invention, there is provided a pharmaceutical composition comprising a polypeptide according to the second aspect of the invention or a primary cell according to the third aspect of the invention, together with a pharmaceutically acceptable carrier or diluent.

We provide, according to a fifth aspect of the present invention, a method of treating or preventing a disease in a patient, the method comprising the steps of: (a) providing a primary cell; (b) introducing a nucleic acid binding polypeptide into the primary cell, in which the nucleic acid binding polypeptide binds to and regulates a nucleic acid sequence responsible for or associated with the disease; and (c) introducing the primary cell into the patient.

Preferably, the primary cell is provided from the patient to be treated.

The present invention, in a sixth aspect, provides a method of expressing a protein in a primary cell, the method comprising the steps of: (a) providing a primary cell comprising a nucleic acid sequence encoding a protein; (b) introducing a nucleic acid binding polypeptide into the primary cell, in which the nucleic acid binding polypeptide binds to and promotes the expression of the protein from the nucleic acid sequence. Preferably, the primary cell is of a cell type which does not normally express the protein.

In a seventh aspect of the present invention, there is provided a method of expressing an exogenous nucleic acid binding polypeptide in a primary cell, the method comprising the steps of: (a) providing a nucleic acid sequence encoding a nucleic acid binding polypeptide operatively linked to a control sequence; (b) introducing the nucleic acid sequence into the primary cell, or an ancestor of the primary cell; and (c) allowing the nucleic acid binding polypeptide to be expressed from the nucleic acid sequence within the primary cell.

### BRIEF DESCRIPTION OF THE FIGURES

FIG. 1 shows repression of TNFR1 receptor expression in HUVEC cells by a zinc finger repression peptide, specifically targeted to the TNFR1 promoter. Expression of TNFR1 on the surface of HUVEC cells expressing the repressor peptide is indicated by the filled area, while the open area represents the expression of TNFR1 on HUVEC cells which do not express a zinc finger repressor targeted to the TNFR1 promoter (these are used as a negative control).

FIG. 2 is a graph showing the relative binding affinity of the EPOb-a-VP64 peptide to its target site, EPO B-A (TCTGGGGTGGGGGCTGGG); control site 1 (TCTGGGGTGGGGGCTAAA); control site 2 (TCTGGGGTGAAAGCTGGG); control site 3 (TCTGGGGTGGCTGGG); and to a no DNA negative control.

FIG. 3A is a standard erythropoietin curve obtained using the Erythropoietin ELISA kit (R&D Systems).

FIG. 3B shows the concentration of erythropoietin secreted by cells transfected with: an empty viral vector; a vector containing a zinc finger peptide which doesn't target the human erythropoietin promoter; a vector containing EPOb-a-VP64.

### DETAILED DESCRIPTION OF THE INVENTION

According to the invention, a nucleic acid sequence in a primary cell may be targeted by the use of one or more nucleic acid binding polypeptides, and expression of the nucleic acid sequence regulated. Expression of the nucleic acid sequence may be up-regulated or down-regulated, according to the condition to be treated.

Preferably, zinc finger polypeptide(s) are used as nucleic acid binding polypeptides, as described in detail elsewhere in this document. This document also describes in detail rules for the design of such fingers capable of binding specific target sequences, as well as methods of selection of such fingers from libraries. The nucleic acid binding polypeptides may comprise one or more regulatory domains, such as transcriptional activator domains, or transcriptional repressor domains, also as described in further detail below.

The target site or sequence bound by the nucleic acid binding polypeptide is preferably in a regulatory region of a gene. The gene to be regulated may be an endogenous cellular gene, by which we mean a gene that is native to a cell, which is in its normal genomic and chromatin context, and which is not heterologous to the cell. However, expression of a heterologous gene (i.e., a gene which is not normally present in the cell but is introduced) may be regulated by the methods described here.

The methods described here are particularly useful in targeting and regulating the expression of a gene which is present in a primary cell. In particular, the methods are useful for regulating genes which are not expressed in or are not expressed at significant levels in the cells as obtained. For example, our methods may be used to turn on expression of developmentally silent or inactive genes. Thus, genes whose expression is repressed or not activated (turned off) in certain cell types, during certain developmental stages of a cell type, during certain time periods in a cell type, or during certain stages of the cell cycle may be turned on, and vice versa. Furthermore, the methods described here are useful for down-regulating genes which are expressed at undesirably high levels in primary cells as obtained.

For example, the methods may be used to turn on genes, such as the human erythropoietin, growth hormone and insulin genes and other genes (e.g., genes encoding Factor VIII, Factor IX, erythropoietin, alpha-1 antitrypsin, calcitonin, glucocerebrosidase, growth hormone, low density lipoprotein (LDL) receptor, IL-2 receptor and its antagonists, insulin, globin, immunoglobulins, catalytic antibodies, the interleukins, insulin-like growth factors, superoxide dismutase, immune response modifiers, parathyroid hormone, interferons, nerve growth factors, tissue plasminogen activators, and colony stimulating factors) in a primary cell. The present methods may in particular be used for gene therapy.

Thus, for example, our methods are useful to down-regulate genes involved in viral infection, for example, down-regulation of receptors involved in viral infection (e.g., CXCR4) in primary cells will decrease the chances of viral infection. Furthermore, genes involved in inflammatory responses, such as IL-1 mediated responses, may be down-regulated to achieve decreased inflammation. Down-regulation of cytokine receptors may be achieved by using nucleic acid binding polypeptides which target and down-regulate expression from cytokine receptor genes. Tumourigenesis may be regulated by targeting expression of oncogenes such as c-myc, c-myb and ras (preferably, down-regulation of oncogene expression), or by targeting expression of tumour suppressor genes (such as p53, retinoblastoma, etc, which are preferably up-regulated). Neurological disorders such as Alzheimer's disease may be treated by regulating the expression of amyloid precursor protein (APP), PS1, PS2, etc. Our invention is also useful in treating or preventing metabolic disorders such as diabetes or obesity, through the regulation of expression of metabolic proteins or regulators such as low density lipoprotein (LDL) or their receptors (such as LDL-receptor, LDL-R).

One or more nucleotide sequences within the control region(s) or regulatory sequence(s) of the genes may be targeted. Such regulatory sequences may be comprised of promoters, enhancers, scaffold-attachment regions, negative regulatory elements, transcriptional initiation sites, regulatory protein binding sites or combinations of these sequences. As a result, an endogenous copy of a gene encoding a desired gene product is turned on (expressed) or off (not expressed, or inhibited). Furthermore, nucleotide sequences within RNA transcripts (for example, ribosome binding regions, or ribosome pause sites) may be targeted.

Primary Cells

As the term is used in this document, “primary cells” means cells which are directly derived from the body of an organism, or clonal descendants of these cells. Thus, primary cells include those in a tissue mass taken from an organism, whether alive or dead, for example, cells in a tissue sample such as a biopsy sample. As used in this document, the term “primary cell” includes both normal as well as preferably transformed (tumour) cells taken from an organism. Primary cells also include cells taken from an organism which have been dissociated for growing in vitro, for example, in a tissue culture flask. In addition, such cells, as well as clonal descendants of such cells growing in culture, for example, in vitro tissue culture are considered primary cells for the purposes of this document.

Thus, primary cells include cells present in a suspension of cells isolated from a vertebrate tissue source (prior to their being plated, i.e., attached to a tissue culture substrate such as a dish or flask), cells present in an explant derived from tissue, both of the previous types of cells plated for the first time, descendants of such cells and cell suspensions derived from these plated cells.

Cells in culture will continue growing until confluence, when contact inhibition causes cessation of cell division and growth. Such cells may then be dissociated from the substrate or flask, and “split” or passaged, by dilution into tissue culture medium and replating. The term “passage” designates the process consisting in taking an aliquot of a confluent culture of a cell line, in inoculating into fresh medium, and in culturing the line until confluence or saturation is obtained. Cell lines are thus traditionally cultured by successive passages in fresh media.

It has been established that “normal” (i.e., untransformed) cells derived directly from an organism are not immortal. In other words, such cells have a limited life span in culture (they are mortal). They will not continue growing indefinitely, but will ultimately lose the ability to proliferate or divide after a certain number of generations. On reaching a “crisis phase” such cells die after about 50 generations. Thus, such cells may only be passaged a limited number of times. Such cells are included within the definition of “primary cells”. A primary cell line therefore includes one which has been derived from normal (i.e. not tumour) primary tissues and maintained in a non-immortalised state for, for example, fewer 50 divisions. Preferably, primary cells include those cultured for fewer than 40 divisions, more preferably, fewer then 30 divisions, fewer then 20 divisions, fewer then 10 divisions, or fewer then 5 divisions. Most preferably, the term “primary cell” is taken to mean cells cultured for 0, 1, 2, 3, 4 or 5 divisions.

It is known that certain treatments may be used in order to immortalise normal, untransformed, cells derived from the body of an organism, and to allow them to continue to divide and proliferate in culture indefinitely. Such treatments include fusion (for example, using PEG) with tumour cells or tumour cell lines. Furthermore, viral infection of a cell line with tranforming viruses such as SV40, EBV, HBV or HTLV-1 may also lead to a transformed or immortal phenotype. Techniques for the transfection of cells, with the aid of specially adapted vectors, such as the SV40 vector comprising a sequence of the large T antigen (R. D. Berry et al., Br. J. Cancer, 57, 287-289, 1988), or a vector comprising DNA sequences of the human papillomavirus (U.S. Pat. No. 5,376,542), are known in the art.

Immortal cell lines may also be created by transfer of dominant oncogenes into primary cells (Chou, J. Y., Mol. Endocrinol., 3:1511-14 (1989)). Such cell lines have been constructed from brain, liver and bone marrow. Furthermore, the combined expression of SV40 T-antigen and hTRT (human telomerase catalytic subunit) may be used to achieve immortalization in human primary skin fibroblasts (Bodnar-A-G. et. al., Science (1998) 279: p. 349-52). Genetic changes may also occur to cells in culture which enable them to become immortal. These genetic changes may arise spontaneously, or may be induced. Such changes may include aneuploidy, mutations such as point mutations, inversions, deletions, insertions, transfection of a suitable DNA construct etc.

The definition of “primary cell” as used in this document specifically does not include normal cells which are derived from the body of an organism, and which have been treated ex-vivo or in vitro to render them immortal, or which undergo other changes in vitro or ex-vivo which lead to a transformed phenotype, nor descendants of such cells (i.e., cells which have been immortalised in vitro or ex vivo). Thus, a “primary cell” is not one which has been transformed ex-vivo or in vitro, whether by fusion with an immortalised cell, by viral infection, by introduction of a dominant oncogene, or by mutation, etc.

The term “clonal descendant” of a cell derived from the body of an organism is therefore preferably to be taken in a strict sense to refer to descendants of the original cells which have not undergone substantially any transforming treatment or genetic alteration. Such clonal descendants have not undergone substantial genomic changes are substantially genetically identical to the parent cell, or an ancestor, preferably, an original cell which was taken from the body of the organism. However, and as noted above, “primary cells” preferably includes transformed, cancer or tumour cells taken from the body of an organism, which already possess a transformed phenotype; descendants of these cells are also included. Such cells may usefully be regarded as having been transformed in vivo to have an immortalised phenotype, and are immortal or transformed as taken from the body of the organism from which they derive. They do not require any subsequent transformation steps in vitro or ex-vivo (as described above) to render them immortal. The term “primary cells” should also preferably be taken to include primary cell lines derived from primary cells.

In vitro culture of cancer or tumour cells (including detail on the culture of specific tumour types, collection and handling, dis-aggregation, tumour cell selection, culture methods, subculture and cloning, and tissue and tumour cell identification) is described in detail in, for example, Human Cancer in Primary Culture: A Handbook (Developments in Oncology, Vol. 64, John R. W. Masters (Editor).

Sources of Primary Cells

The primary cell which is to be targeted may be obtained from a variety of tissues and include all cell types which can be maintained in culture.

For example, primary cells which may be regulated by the present method include fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells, hepatocytes and precursors of these somatic cell types. As described below, primary cells may be targeted and put into an organism. Primary cells are preferably obtained from the individual to whom the transfected primary cells is administered. However, primary cells may be obtained from a donor (other than the recipient) of the same species or another species (e.g., nonhuman primates, mouse, rat, rabbit, cat, dog, pig, cow, bird, sheep, goat, horse). Methods of obtaining and culturing primary cells are known in the art, and are also described in detail below.

Preferably, the primary cells which may be targeted using the methods described here include those shown in Table 1, for example.

TABLE 1
Primary cells, cell lines and their characteristics

| Name (ATCCno., | | | |
|---|---|---|---|
| ECACCno. | | | |
| or reference) | Tissue | Morphology | Species |
| Angioblasts | peripheral blood | | human |
| ASMC | aorta | smooth muscle | human |
| (Yan et al, (2000) J. | | | |
| Biol. Chem. 275: | | | |
| 4949-4955) | | | |
| Astrocytes | | | rat |
| Bovine aortic | aorta | endothelial | bovine |
| endothelial cells | | | |
| Cardiomyocytes | | | rat |
| Chondrocytes | | | mouse |
| Chromaffin cells | adrenal medulla | | bovine |
| Endothelial cells, | cardiac | endothelial | human |
| cardiac | | | |
| Endothelial cells, | coronary aterial | endothelial | human |
| coronary aterial | | | |
| Epithelial cells, | mammary | epithelial | human |
| mammary | | | |
| Epithelial cells, | prostate | epithelial | human |
| prostate | | | |
| Epithelial cells, | tracheal | epithelial | rat |
| tracheal | | | |
| Fibroblasts | not known | | not known |
| Fibroblasts, embryonic | embryonic | | chicken |
| Fibroblasts, forskin | forskin | | human |
| (from Invitrocyte | | | |
| (Seattle, Washington) | | | |
| Fibroblasts, skin | skin | | human |
| Hepatocytes | liver | | |
| HSMC | umbilical vein | smooth muscle | human |
| HUVEC endothelial | umbilical vein | endothelial | human |
| cells (no number) | | | |
| Keratinocytes | | epithelial | human |
| Keratinocytes | skin | epithelial | mouse |
| (Vasioukhin et al, | | | |
| (2000) Cell 100: | | | |
| 209-219) | | | |
| Keratinocytes, foreskin | forskin | epithelial | human |
| MEF TRP53-/- | carcass | fibroblasts | mouse |
| Mepg2 | | | |
| Muscle cells, | embryonic leg | | chicken |
| embryonic leg | | | |
| Myoblast | muscle | differentiated 5-7 | mouse |
| | | days, fused | |
| | | myotubes | |
| Myoblasts, | sceletal muscle | | canine |
| sceletal muscle | | | |
| Myocyte, cardiac | Cardiac, heart | | mouse |
| Neurons | cerebellar, brain | | mouse |
| Oligodendrocytes | | | rat |
| PAE | aorta | endothelial | pig |
| (Jefferies et al, (2000) | | | |
| J. Biol. Chem. 275: | | | |
| 2877) | | | |
| PVEC | lung vein | epithelial | rat |
| (Tang et al, (2000) J. | | | |
| Biol. Chem. 275: | | | |
| 8389-8396) | | | |
| Retinal pigment | eye | epithelial | human |
| epithelial cells (hRPE) | | | |
| Satellite | muscle | | human |
| Sensory epithelial cells | inner-ear | epithelial | chicken |
| Sertoli | testes | | rat |
| Smooth muscle cells, | coronary artery | | pig |
| coronary artery | | | |
| Smooth muscle cells, | jejunum | | human |
| jejunum | | | |
| Testes cells | testes | | lamb |
| Thyroid cells | | | human |
| Uterine Stromal | uterus | epithelial | human |

Primary cells may also be obtained from cell culture collections such as the American Type Culture Collection (ATCC); furthermore, commercially available sources of primary cells may be used. For example, human vascular endothelial cells obtained from umbilical vein (HUVEC) are offered in various forms by Clonetics Corporation (Walkersville, Md. USA). Primary cells may, as mentioned above, be obtained directly from biopsy of an organism (e.g., a patient).

For example, primary human fibroblasts can be obtained from a variety of tissues, including biopsy specimens derived from liver, kidney, lung and skin. The isolation of primary skin fibroblasts, which are readily obtained from individuals of any age with minimal discomfort and risk are described here in detail (primary embryonic and foetal fibroblasts may be isolated using this protocol as well). Minor modifications to the protocol can be made if the isolation of fibroblasts from-other tissues is desired.

Human skin is obtained following circumcision or punch biopsy. The specimen consists of three major components: the epidermal and dermal layers of the skin itself, and a fascial layer that adheres to the dermal layer. Fibroblasts can be isolated from either the dermal or fascial layers. Approximately 3 cm² tissue is placed into approximately 10 ml of wash solution (Hank's Balanced Salt Solution containing 100 units/ml penicillin G, 100 μg/ml streptomycin sulfate, and 0.5 μg/ml Fungisone) and subjected to gentle agitation for a total of three 10-minute washes at room temperature. The tissue is then transferred to a 100 mm tissue culture dish containing 10 ml digestion solution (wash solution containing 0.1 units/ml collagenase A, 2.4 units/ml grade II Dispase).

Under a dissecting microscope, the skin is adjusted such that the epidermis is facing down. The fascial tissue is separated from the dermal and epidermal tissue by blunt dissection. The fascial tissue is then cut into small fragments (less than 1 mm²) and incubated on a rotating platform for 30 min at 37 degrees C. The enzyme/cell suspension is removed and saved, an additional 10 cc of digestion solution is added to the remaining fragments of tissue, and the tissue is reincubated for 30 min at 37 degrees C. The enzyme/cell suspensions are pooled, passed through a 15-gauge needle several times, and passed through a Cellector Sieve (Sigma) fitted with a 150-mesh screen. The cell suspension is centrifuged at 1100 rpm for 15 min at room temperature. The supernatant is aspirated and the disaggregated cells resuspended in 10 ml of nutrient medium (see below). Primary fibroblast cultures are initiated on tissue culture treated flasks (Coming) at a density of approximately 40,000 cells/cm².

Isolation of human dermal fibroblasts may also be achieved as follows: Fascia is removed from skin biopsy or circumcision specimen as described above and the skin is cut into small fragments less than 0.5 cm². The tissue is incubated with 0.25% trypsin for 60 min at 37 degrees C. (alternatively, the tissue can be incubated in trypsin for 18 hrs at 4 degrees C.). Under the dissecting microscope, the dermis and epidermis are separated. Dermal fibroblasts are then isolated as described above for fascial fibroblasts. The procedure is essentially as described above. Skin should be removed from areas that have been shaved and washed with a germicidial solution and surgically prepared using accepted procedures.

Culturing of the isolated primary human fibroblasts is described here (further procedures for culture of primary cells are covered in the next section). When confluent, the primary culture is trypsinized using standard methods and seeded at approximately 10,000 cells/cm². The cells are cultured at 37 degrees C. in humidified air containing 5% CO₂. Human fibroblast nutrient medium (containing DMEM, high glucose with sodium pyruvate, 10-15% calf serum, 20 mM HEPES, 20 mM L-glutamine, 50 units/ml penicillin G, and 10.mu.g/ml streptomycin sulfate) is changed twice weekly.

According to the methods described here, nucleic acid sequences within primary cells may be regulated by introducing a suitable nucleic acid binding polypeptide such as a zinc finger into the primary cell, or to an ancestor of the cell.

Transfection of primary cells for introduction of zinc finger coding constructs may be achieved by means known in the art. Suitably, an expression construct capable of expressing the zinc finger nucleic acid binding polypeptide is transfected into the primary cell or an ancestor. Various expression constructs suitable for transfection of nucleic acid binding polypeptide sequences are known in the art, and are described in further detail elsewhere in this document. Such constructs may be transfected by use of for example, calcium phosphate and DEAE mediated transfection; furthermore liposome mediated transfection may be achieved.

Gene transfer into primary islet cells has been accomplished by electroporation (German, M. S., et al., J. Biol. Chem., 265:22063-22066 (1990)); furthermore, adenovirus vectors have been found to efficiently infect pancreatic cells (Newgard, C. B., Diabetes, 43:341-50 (1994)). The monocationic chemical DOTAP (Roche Molecular Biochemicals) comprises a liposome formulation and may be used for the cationic liposome-mediated transfection of negatively charged molecules into eukaryotic cells. Another liposomal formulation based on the polycationic chemical DOSPER (Roche Molecular Biochemicals) may be used for the liposome-mediated transfer of DNA, RNA, and other negatively charged molecules into eukaryotic cells. Noah et al, 1998, Biochemica 2, 38-40 describe the transfection of primary cardiac myocyte cultures with dna and anti-sense oligonucleotides using FuGENE (Roche Molecular Biochemicals). Tranfection reagents such as FuGENE have been found to be useful in transfection of the primary cells listed in Table 1.

Transfection of Peripheral Blood Lymphocytes (PBLs) may be achieved by use of retroviral vectors carrying a nucleic acid sequence encoding a relevant nucleic acid binding polypeptide such as a zinc finger. Furthermore, agents are commercially available which enable transfection of plasmid DNA to be achieved (for example, the Effectene and Superfect reagents from Qiagen).

Culture of Primary Cells

Tissue culture of primary cells is known in the art. Generally, culture conditions will depend on the type of primary cell chosen. Reference is made to Human Cancer in Primary Culture: A Handbook (Developments in Oncology, Vol. 64, John R. W. Masters (Editor).

As an example, we provide a protocol for culture, freezing and storage of primary Breast Epithelial Cells, as described in http://qcom.etsu.edu/biochem/protocols/epithelial.htm. Cells may be obtained from individuals and frozen down from 60 mm plates containing about 1×10⁵ cells and stored in liquid nitrogen. On thawing, such cells need to be grown in a 1% CO₂ incubator.

The tissue culture medium needs to be supplemented with various growth factors, including: MEBM-SBF (for example, from Clonetics, cat# CC-3152), Human Epidermal Growth Factor (for example, from Upstate Biotechnology), Hydrocortizone (for example, from Sigma, cat# H4001), Insulin (for example, from Sigma, cat# 1-5500), Bovine Pituitary Extract (for example, from Clonetics, cat# CC-4009), Transferrin, Human (for example, from Sigma, cat# T-2252), Isoprotemol (for example, from Sigma, cat# 1-5627). These growth factors may be to be aliquoted into stock solutions: (a) make a 20,000× stock solution of HEGF by adding 1 ml of sterile dH₂O to the 100 μg vial of EGF. For a 500 ml bottle of MEBM media, use 2511 of the 20,000× stock; (b) make a 2000× stock of hydrocortizone by adding 50 mg of hydrocortizone to 50 ml of 95% ethanol (or 1 mg/ml). Mix well. For a 500 ml bottle of media, use 250 μl of the 2000× stock; (c) make a 200× stock solution of insulin by dissolving 1 g of insulin in 200 ml of 0.005 N HCl, need to stir. Then bring up the solution to 1 litre by adding 800 ml of sterile dH₂O. This makes a final concentration of 1 mg/ml. Filter sterilize. For a 500 ml bottle of media, use 2.5 ml of the 200× stock solution; (d) bovine Pituitary Extract aliqoted into 35 mg samples or a 1× stock (will need 2.69 ml aliquots for the 500 mls of media); (e) make a 2000× stock of transferrin by dissolving 1000 mg of transferrin in 100 ml of sterile dH₂O, this gives a 10 mg/ml stock. Filter sterilize through a 0.2 μ filter. For a 500 ml bottle of media, use 250 μl of the stock solution; (f) make a 500× stock of isoproternol by making 40 mls of 0.05M isoproternol in 95% ethanol (use 50 mg of isoproternol for 40 mls of 95% EtOH). For a 500 ml bottle of MEBM media, use 1 ml of the 500× stock.

To prepare 500 mls of MEBM media complete with all of the above growth factors, 6.715 mls of media are removed in a sterile hood. The growth factors are added back as follows: 25 μl of EGF (20,000×) for a final concentration of 5 ng/ml; 250 μl of Hydrocortizone (2000×) for a final concentration of 0.5 μg/ml; 2.5 ml of Insulin (200×) for a final concentration of 5 μg/ml; 2.69 ml of BPE (1×) for a final concentration of 70 μg/ml; 250 μl of Transferrin (2000×) for a final concentration of 5 μg/ml; 1.0 ml of Isoproternol (500×) for a final concentration of 0.00010 M.

In order to thaw cells, primary cells are removed from liquid nitrogen and placed on dry ice. Suspend the cryotube of primary cells in a 37 degrees C. water bath (do not immerse). Once thawed, wipe down the tube with 70% ethanol. Add 500 μl of the supplemented media to the tube, and gently resuspend the primary cells. The primary cell suspension is transferred to a 60 mm plate; and brought up to a final volume of 5 mls with the supplemented media. The primary cells are placed in a 1% CO₂, 37 degrees C. incubator overnight. The media is aspirated off and replaced with 5 mls of fresh media. Place in incubator overnight. Primary cells should begin to grow well in about two to three days.

In order to split or farm primary cells when they become confluent, 0.05% Trypsin with 0.02% EDTA is used. The medium is aspirated off and primary cells washed once with 3 mls of trypsin. The trypsin is aspirated off and more trypsin (Oust enough to cover the cells, about 600 μL for a 60 mm plate) is added. The plates are put back in the incubator for about 3-5 minutes. 1 ml of PBS is then added to each plate, and primary cells resuspended using a pasteur pipet. The primary cells are collected in a 15 ml conical tube, and spun at setting three in a clinical centrifuge for about five minutes. PBS is then aspirated off and the primary cells resuspended with about 1 ml of media using a pasteur pipet. Primary cells are transfered to a 60 mm plate by splitting them the desired proportion, and the total volume on the plate brought up to 5 mls. The passage number is marked on the plates.

In order to freeze primary cells for storage, 10% Glycerol, 15% Fetal Calf Serum, and 75% MEBM base media is used as the freezing medium (stored at −20 degrees C. until ready to use). Once thawed the medium should be stored at 4 degrees C. Medium is aspirated off from the plate, and the cells washed once with 3 mls trypsin, and then aspirate off. 600 μl of trypsin is further added and the plate placed in the incubator for about 3-5 min. 1 ml of PBS is added to resuspend cells, and the cells transferred to 15 ml conical tubes and spun at setting three for about 5 min. The PBS is removed and the cells resuspended with 1 ml of freezing media. Cells are transferred to a cryovial labelled with the date, the cell type, and the passage number. Cells are placed in a styrofoam container and kept at −70 degrees C. for 24 hrs to prevent shock. After 24 hrs at −70 degrees C., cells are moved to liquid nitrogen.

Cell-strains and primary cells may also be grown on microcarriers in homogeneous culture, as described in Van Wezel (Nature, 216:64-65, 1967).

Selectable Markers

A variety of selectable markers may be incorporated into the primary cell. For example, a selectable marker which confers a selectable phenotype such as drug resistance, nutritional auxotrophy, resistance to a cytotoxic agent or expression of a surface protein, may be used. Selectable marker genes which can be used include neo, gpt, dhfr, ada, pac, hyg, mdrl and hisD. The selectable phenotype conferred makes it possible to identify and isolate recipient primary cells.

Selectable markers may be divided into two categories: positive selectable and negative selectable. In positive selection, cells expressing the positive selectable marker are capable of surviving treatment with a selective agent (such as neo, gpt, dhfr, ada, pac, hyg, mdr1 and hisD). In negative selection, cells expressing the negative selectable marker are destroyed in the presence of the selective agent (e.g., tk, gpt).

For the purposes of gene therapy, the primary cells used may generally be patient-specific genetically-engineered cells. It is possible, however, to obtain cells from another individual of the same species or from a different species. Use of such cells may require administration of an immuno-suppressant, alteration of histocompatibility antigens, or use of a barrier device to prevent rejection of the implanted cells. For many diseases, this will be a one-time treatment and, for others, multiple gene therapy treatments will be required.

Nucleic Acid Binding Polypeptides

We describe in this document regulation of gene expression in primary cells using nucleic acid binding polypeptides.

The term “polypeptide” (and the terms “peptide” and “protein”) are used interchangeably to refer to a polymer of amino acid residues, preferably including naturally occurring amino acid residues. Artificial analogues of amino acids may also be used in the nucleic acid binding polypeptides, to impart the proteins with desired properties or for other reasons. The term “amino acid”, particularly in the context where “any amino acid” is referred to, means any sort of natural or artificial amino acid or amino acid analogue that may be employed in protein construction according to methods known in the art. Moreover, any specific amino acid referred to herein may be replaced by a functional analogue thereof, particularly an artificial functional analogue. Polypeptides may be modified, for example by the addition of carbohydrate residues to form glycoproteins.

As used herein, “nucleic acid” includes both RNA and DNA, constructed from natural nucleic acid bases or synthetic bases, or mixtures thereof. Preferably, however, the binding polypeptides of the invention are DNA binding polypeptides.

Zinc Fingers

Particularly preferred examples of nucleic acid binding polypeptides are Cys2-His2 zinc finger binding proteins which, as is well known in the art, bind to target nucleic acid sequences via α-helical zinc metal atom co-ordinated binding motifs known as zinc fingers. Each zinc finger in a zinc finger nucleic acid binding protein is responsible for determining binding to a nucleic acid triplet, or an overlapping quadruplet, in a nucleic acid binding sequence. Preferably, there are 2 or more zinc fingers, for example 2, 3, 4, 5, 6, 7, 8, 9, 10, 11, 12, 13, 14, 15, 16, 17, 18 or more zinc fingers, in each binding protein. Advantageously, the number of zinc fingers in each zinc finger binding protein is a multiple of 2.

All of the DNA binding residue positions of zinc fingers, as referred to herein, are numbered from the first residue in the α-helix of the finger, ranging from +1 to +9. “−1” refers to the residue in the framework structure immediately preceding the CL-helix in a Cys2-His2 zinc finger polypeptide. Residues referred to as “++” are residues present in an adjacent (C-terminal) finger. Where there is no C-terminal adjacent finger, “++” interactions do not operate.

The present invention is in one aspect concerned with the production of what are essentially artificial DNA binding proteins. In these proteins, artificial analogues of amino acids may be used, to impart the proteins with desired properties or for other reasons. Thus, the term “amino acid”, particularly in the context where “any amino acid” is referred to, means any sort of natural or artificial amino acid or amino acid analogue that may be employed in protein construction according to methods known in the art. Moreover, any specific amino acid referred to herein may be replaced by a functional analogue thereof, particularly an artificial functional analogue. The nomenclature used herein therefore specifically comprises within its scope functional analogues or mimetics of the defined amino acids.

The α-helix of a zinc finger binding protein aligns antiparallel to the nucleic acid strand, such that the primary nucleic acid sequence is arranged 3′ to 5′ in order to correspond with the N terminal to C-terminal sequence of the zinc finger. Since nucleic acid sequences are conventionally written 5′ to 3′, and amino acid sequences N-terminus to C-terminus, the result is that when a nucleic acid sequence and a zinc finger protein are aligned according to convention, the primary interaction of the zinc finger is with the -strand of the nucleic acid, since it is this strand which is aligned 3′ to 5′. These conventions are followed in the nomenclature used herein. It should be noted, however, that in nature certain fingers, such as finger 4 of the protein GLI, bind to the + strand of nucleic acid: see Suzuki et al., (1994) NAR 22:3397-3405 and Pavletich and Pabo, (1993) Science 261:1701-1707. The incorporation of such fingers into DNA binding molecules according to the invention is envisaged.

Engineering, Rational and Rule Based Design of Zinc Fingers

The present invention may be integrated with the rules set forth for zinc finger polypeptide design in our European or PCT patent applications having publication numbers; WO 98/53057, WO 98/53060, WO 98/53058, WO 98/53059, describe improved techniques for designing zinc finger polypeptides capable of binding desired nucleic acid sequences. In combination with selection procedures, such as phage display, set forth for example in WO 96/06166, these techniques enable the production of zinc finger polypeptides capable of recognising practically any desired sequence.

We therefore describe a method for regulating a gene in a primary cell, the method comprising providing a control sequence of a gene comprising a nucleic acid quadruplet, preparing a nucleic acid binding protein of the Cys2-His2 zinc finger class capable of binding to the nucleic acid quadruplet, and allowing the nucleic acid binding protein to bind to the nucleic acid quadruplet in a primary cell, wherein binding to each base of the quadruplet by an α-helical zinc finger nucleic acid binding motif in the protein is determined as follows:

(a) if base 4 in the quadruplet is G, then position +6 in the α-helix is Arg or Lys;

(b) if base 4 in the quadruplet is A, then position +6 in the α-helix is Glu, Asn or Val; (c) if base 4 in the quadruplet is T, then position +6 in the α-helix is Ser, Thr, Val or Lys; (d) if base 4 in the quadruplet is C, then position +6 in the α-helix is Ser, Thr, Val, Ala, Glu or Asn; (e) if base 3 in the quadruplet is G, then position +3 in the α-helix is His; (f) if base 3 in the quadruplet is A, then position +3 in the α-helix is Asn; (g) if base 3 in the quadruplet is T, then position +3 in the α-helix is Ala, Ser or Val; provided that if it is Ala, then one of the residues at −1 or +6 is a small residue; (h) if base 3 in the quadruplet is C, then position +3 in the α-helix is Ser, Asp, Glu, Leu, Thr or Val; (i) if base 2 in the quadruplet is G, then position −1 in the α-helix is Arg; 0) if base 2 in the quadruplet is A, then position −1 in the α-helix is Gln; (k) if base 2 in the quadruplet is T, then position −1 in the α-helix is His or Thr; (1) if base 2 in the quadruplet is C, then position −1 in the α-helix is Asp or His; (m) if base 1 in the quadruplet is G, then position +2 is Glu; (n) if base 1 in the quadruplet is A, then position +2 Arg or Gin; (O) if base 1 in the quadruplet is C, then position +2 is Asn, Gin, Arg, His or Lys; (p) if base 1 in the quadruplet is T, then position +2 is Ser or Thr.

We further describe a method for regulating a gene in a primary cell, the method comprising the steps of providing a control sequence of a gene comprising a nucleic acid quadruplet, preparing a nucleic acid binding protein of the Cys2-His2 zinc finger class capable of binding to the nucleic acid quadruplet, and allowing-the nucleic acid binding protein to bind to the nucleic acid quadruplet in a primary cell, wherein binding to each base of the quadruplet by an α-helical zinc finger nucleic acid binding motif in the protein is determined as follows:

(a) if base 4 in the quadruplet is G, then position +6 in the α-helix is Arg; or position +6 is Ser or Thr and position ++2 is Asp; (b) if base 4 in the quadruplet is A, then position +6 in the α-helix is Gin and ++2 is not Asp; (c) if base 4 in the quadruplet is T, then position +6 in the α-helix is Ser or Thr and position ++2 is Asp; (d) if base 4 in the quadruplet is C, then position +6 in the α-helix may be any amino acid, provided that position ++2 in the α-helix is not Asp; (e) if base 3 in the quadruplet is G, then position +3 in the α-helix is His; (f) if base 3 in the quadruplet is A, then position +3 in the α-helix is Asn; (g) if base 3 in the quadruplet is T, then position +3 in the α-helix is Ala, Ser or Val; provided that if it is Ala, then one of the residues at −1 or +6 is a small residue; (h) if base 3 in the quadruplet is C, then position +3 in the α-helix is Ser, Asp, Glu, Leu, Thr or Val; (i) if base 2 in the quadruplet is G, then position −1 in the α-helix is Arg; (j) if base 2 in the quadruplet is A, then position −1 in the α-helix is Gln; (k) if base 2 in the quadruplet is T, then position −1 in the α-helix is Asn or Gln; (l) if base 2 in the quadruplet is C, then position −1 in the α-helix is Asp; (m) if base 1 in the quadruplet is G, then position +2 is Asp; (n) if base 1 in the quadruplet is A, then position +2 is not Asp; (o) if base 1 in the quadruplet is C, then position +2 is not Asp; (p) if base 1 in the quadruplet is T, then position +2 is Ser or Thr.

The foregoing represents sets of rules which permits the design of a zinc finger binding protein specific for any given target DNA sequence. Such zinc finger binding proteins are capable of being used to down-regulate or up-regulate expression of one or more genes in a primary cell. A zinc finger binding motif is a structure well known to those in the art and defined in, for example, Miller et al., (1985) EMBO J. 4:1609-1614; Berg (1988) PNAS (USA) 85:99-102; Lee et al., (1989) Science 245:635-637; see International patent applications WO 96/06166 and WO 96/32475, corresponding to U.S. Ser. No. 08/422,107, incorporated herein by reference.

In general, a preferred zinc finger framework has the structure:

X₀₋₂CX₁₋₅CX₉₋₁₄H X₃₋₆H/c

where X is any amino acid, and the numbers in subscript indicate the possible numbers of residues represented by X (Formula A).

The above framework may be further refined to include the structure:

X₀₋₂C X₁₋₅C X₂₋₇X X X X X X X H X₃₋₆H/c −1 1 2 3 4 5 6 7 (A′)

where X is any amino acid, and the numbers in subscript indicate the possible numbers of residues represented by X (Formula A′).

In a preferred aspect of the present invention, zinc finger nucleic acid binding motifs may be represented as motifs having the following primary structure:

X^{a}C X₂₋₄C X₂₋₃F X^{c}X X X X L X X H X X X^{b}H—linker −1 1 2 3 4 5 6 7 8 9 (B)

wherein X (including X^{a}, X^{b} and X^{c}) is any amino acid. X₂₋₄ and X₂₋₃ refer to the presence of 2 or 4, or 2 or 3, amino acids, respectively (Formula B).

The Cys and His residues, which together co-ordinate the zinc metal atom, are marked in bold text and are usually invariant, as is the Leu residue at position +4 in the α-helix.

The linker may comprise a canonical, structured or flexible linker. Structured and flexible linkers (as well as canonical linkers) are described elsewhere in this document, and in our UK application numbers GB 0001582.6, GB0013103.7, GB0013104.5 and our International Patent Application PCT/GB00/00202, all of which are hereby incorporated by reference.

Modifications to this representation may occur or be effected without necessarily abolishing zinc finger function, by insertion, mutation or deletion of amino acids. For example it is known that the second His residue may be replaced by Cys (Krizek et al., (1991) J. Am. Chem. Soc. 113:4518-4523) and that Leu at +4 can in some circumstances be replaced with Arg. The Phe residue before X_{c} may be replaced by any aromatic other than Trp. Moreover, experiments have shown that departure from the preferred structure and residue assignments for the zinc finger are tolerated and may even prove beneficial in binding to certain nucleic acid sequences. Even taking this into account, however, the general structure involving an α-helix co-ordinated by a zinc atom which contacts four Cys or His residues, does not alter. As used herein, structures (A), (A′) and (B) above are taken as an exemplary structure representing all zinc finger structures of the Cys2-His2 type.

Preferably, X^{a} is F/γ-X or P-F/γ-X. In this context, X is any amino acid. Preferably, in this context X is E, K, T or S. Less preferred but also envisaged are Q, V, A and P. The remaining amino acids remain possible.

Preferably, X₂₋₄ consists of two amino acids rather than four. The first of these amino acids may be any amino acid, but S, E, K, T, P and R are preferred. Advantageously, it is P or R. The second of these amino acids is preferably E, although any amino acid may be used.

Preferably, X^{b} is T or I. Preferably, X^{c} is S or T.

Preferably, X₂₋₃ is G-K-A, G-K-C, G-K-S or G-K-G. However, departures from the preferred residues are possible, for example in the form of M-R—N or M-R.

As set out above, the major binding interactions occur with amino acids −1, +3 and +6. Amino acids +4 and +7 are largely invariant. The remaining amino acids may be essentially any amino acids. Preferably, position +9 is occupied by Arg or Lys. Advantageously, positions +1, +5 and +8 are not hydrophobic amino acids, that is to say are not Phe, Trp or Tyr. Preferably, position ++2 is any amino acid, and preferably serine, save where its nature is dictated by its role as a ++2 amino acid for an N-terminal zinc finger in the same nucleic acid binding molecule.

The code provided by the present invention is not entirely rigid; certain choices are provided. For example, positions +1, +5 and +8 may have any amino acid allocation, whilst other positions may have certain options: for example, the present rules provide that, for binding to a central T residue, any one of Ala, Ser or Val may be used at +3. In its broadest sense, therefore, the present invention provides a very large number of proteins which are capable of binding to every defined target DNA triplet.

Preferably, however, the number of possibilities may be significantly reduced. For example, the non-critical residues +1, +5 and +8 may be occupied by the residues Lys, Thr and Gln respectively as a default option. In the case of the other choices, for example, the first-given option may be employed as a default. Thus, the code according to the present invention allows the design of a single, defined polypeptide (a “default” polypeptide) which will bind to its target triplet. Zinc fingers may be based on naturally occurring zinc fingers and consensus zinc fingers.

In general, naturally occurring zinc fingers may be selected from those fingers for which the DNA binding specificity is known. For example, these may be the fingers for which a crystal structure has been resolved: namely Zif 268 (Elrod-Erickson et al., (1996) Structure 4:1171-1180), GLI (Pavletich and Pabo, (1993) Science 261:1701-1707), Tramtrack (Fairall et al., (1993) Nature 366:483-487) and YY1 (Houbaviy et al., (1996) PNAS (USA) 93:13577-13582). Preferably, the modified nucleic acid binding polypeptide is derived from Zif 268, GAC, or a Zif-GAC fusion comprising three fingers from Zif linked to three fingers from GAC. By “GAC-clone”, we mean a three-finger variant of ZIF268 which is capable of binding the sequence GCGGACGCG, as described in Choo & Klug (1994), Proc. Natl. Acad. Sci. USA, 91, 11163-11167.

The naturally occurring zinc finger 2 in Zif 268 makes an excellent starting point from which to engineer a zinc finger and is preferred.

Consensus zinc finger structures may be prepared by comparing the sequences of known zinc fingers, irrespective of whether their binding domain is known. Preferably, the consensus structure is selected from the group consisting of the consensus structure P Y K C P E C G K S F S Q K S D L V K H Q R T H T, and the consensus structure P Y K C S E C G K A F S Q K S N L T R H Q R I H T.

The consensuses are derived from the consensus provided by Krizek et al., (1991) J. Am. Chem. Soc. 113: 4518-4523 and from Jacobs, (1993) PhD thesis, University of Cambridge, UK. In both cases, canonical, structured or flexible linker sequences, as described below, may be formed on the ends of the consensus for joining two zinc finger domains together.

When the nucleic acid specificity of the model finger selected is known, the mutation of the finger in order to modify its specificity to bind to the target DNA may be directed to residues known to affect binding to bases at which the natural and desired targets differ. Otherwise, mutation of the model fingers should be concentrated upon residues −1, +3, +6 and ++2 as provided for in the foregoing rules.

In order to produce a binding protein having improved binding, moreover, the rules provided by the present invention may be supplemented by physical or virtual modelling of the protein/DNA interface in order to assist in residue selection.

The above rules allow the engineering of a zinc finger capable of binding to a given nucleotide sequence. Engineering of zinc fingers which involves applying rules which specify the choice of amino acid residues based on the identity of residues in a target nucleic acid sequence is referred to here as “rule based” or “rational” design. Such rational design provides a great deal of versatility in zinc finger design.

Selection of Zinc Fingers from Libraries

The rational design described above may be used instead of, or to complement zinc finger production by selection from libraries.

We further describe a method of producing a nucleic acid binding polypeptide capable of regulating gene expression in a primary cell, the method comprising: a) providing a nucleic acid library encoding a repertoire of zinc finger domains or modules, the nucleic acid members of the library being at least partially randomised at one or more of the positions encoding residues −1, 2, 3 and 6 of the α-helix of the zinc finger modules; b) displaying the library in a selection system and screening it against a target DNA sequence comprising a control sequence for the gene; and c) isolating the nucleic acid members of the library encoding zinc finger modules or domains capable of binding to the target sequence. A method of regulating gene expression in a primary cell comprises providing a zinc finger polypeptide produced by the above method, and allowing the zinc finger polypeptide to bind to the target DNA sequence.

The term “library” is used according to its common usage in the art, to denote a collection of polypeptides or, preferably, nucleic acids encoding polypeptides. Methods for the production of libraries encoding randomised members such as polypeptides are known in the art and may be applied in the present invention. The members of the library may contain regions of randomisation, such that each library will comprise or encode a repertoire of polypeptides, wherein individual polypeptides differ in sequence from each other. The same principle is present in virtually all libraries developed for selection, such as by phage display.

Randomisation, as used herein, refers to the variation of the sequence of the polypeptides which comprise the library, such that various amino acids may be present at any given position in different polypeptides. Randomisation may be complete, such that any amino acid may be present at a given position, or partial, such that only certain amino acids are present. Preferably, the randomisation is achieved by mutagenesis at the nucleic acid level, for example by synthesising novel genes encoding mutant proteins and expressing these to obtain a variety of different proteins. Alternatively, existing genes can be themselves mutated, such by site-directed or random mutagenesis, in order to obtain the desired mutant genes.

Zinc finger polypeptides may be designed which specifically bind to nucleic acids incorporating the base U, in preference to the equivalent base T.

In a further preferred aspect, the invention comprises a method of producing a nucleic acid binding polypeptide capable of regulating a gene in a primary cell, the method comprising the steps of: a) providing a nucleic acid library encoding a repertoire of zinc finger polypeptides each possessing more than one zinc finger, the nucleic acid members of the library being at least partially randomised at one or more of the positions encoding residues −1, 2, 3 and 6 of the α-helix in a first zinc finger and at one or more of the positions encoding residues −1, 2, 3 and 6 of the α-helix in a further zinc finger of the zinc finger polypeptides; b) displaying the library in a selection system and screening it against a target DNA sequence comprising a control sequence for the gene; and d) isolating the nucleic acid members of the library encoding zinc finger polypeptides capable of binding to the target sequence. A method of regulating gene expression in a primary cell comprises providing a zinc finger polypeptide produced by the above method, and allowing the zinc finger polypeptide to bind to the target DNA sequence.

In this aspect, the invention encompasses library technology described in our International patent application WO 98/53057, incorporated herein by reference in its entirety. WO 98/53057 describes the production of zinc finger polypeptide libraries in which each individual zinc finger polypeptide comprises more than one, for example two or three, zinc fingers; and wherein within each polypeptide partial randomisation occurs in at least two zinc fingers. This allows for the selection of the “overlap” specificity, wherein, within each triplet, the choice of residue for binding to the third nucleotide (read 3′ to 5′ on the + strand) is influenced by the residue present at position +2 on the subsequent zinc finger, which displays cross-strand specificity in binding. The selection of zinc finger polypeptides incorporating cross-strand specificity of adjacent zinc fingers enables the selection of nucleic acid binding proteins more quickly, and/or with a higher degree of specificity than is otherwise possible.

Zinc finger binding motifs designed according to the invention may be combined into nucleic acid binding polypeptide molecules having a multiplicity of zinc fingers. Preferably, the proteins have at least two zinc fingers. The presence of at least three zinc fingers is preferred. Nucleic acid binding proteins may be constructed by joining the required fingers end to end, N-terminus to C-terminus, with canonical, flexible or structured linkers, as described below. Preferably, this is effected by joining together the relevant nucleic acid sequences which encode the zinc fingers to produce a composite nucleic acid coding sequence encoding the entire binding protein.

The invention therefore provides a method for producing a DNA binding protein as defined above, wherein the DNA binding protein is constructed by recombinant DNA technology, the method comprising the steps of: preparing a nucleic acid coding sequence encoding a plurality of zinc finger domains or modules defined above, inserting the nucleic acid sequence into a suitable expression vector; and expressing the nucleic acid sequence in a host organism in order to obtain the DNA binding protein. A “leader” peptide may be added to the N-terminal finger. Preferably, the leader peptide is MAEEKP.

Multifinger Polypeptides

According to a preferred embodiment of the present invention, the nucleic acid binding polypeptides comprise a plurality of binding domains or motifs. For example, a preferred zinc finger polypeptide according to the invention comprises 2, 3, 4, 5, 6, 7, 8, 9, 10, 11, 12, 13, 14, 15, 16, 17, 18, 19, 20, 21, 22, 23, 24, etc or more zinc finger binding domains or motifs. Highly preferred embodiments are zinc finger polypeptides which comprise three zinc finger motifs and those which comprise six finger motifs.

Zinc finger polypeptides comprising multiple fingers may be constructed by joining together two or more zinc finger polypeptides (which may themselves be selected using phage display, as described elsewhere in this document) with suitable linker sequences. Preferred linker sequences comprise flexible linkers, structured linkers, combined linkers or any combination of these, as described in further detail below.

Means of joining polypeptide sequences, for example, by recombinant DNA technology are known in the art, and are for example disclosed in Sambrook et al (supra) and Ausubel et al (supra). Furthermore, other sequences such as nuclear localisation sequences and “tag” sequences for purification may be included as known in the art.

Flexible and Structured Linkers

The nucleic acid binding polypeptides according to the invention may comprise one or more linker sequences. The linker sequences may comprise one or more flexible linkers, one or more structured linkers, or any combination of flexible and structured linkers. Such linkers are disclosed in our co-pending British Patent Application Numbers 0001582.6, 0013102.9, 0013103.7, 0013104.5 and International Patent Application Number PCT/GB01/00202, which are incorporated by reference.

By “linker sequence” we mean an amino acid sequence that links together two nucleic acid binding modules. For example, in a “wild type” zinc finger protein, the linker sequence is the amino acid sequence lacking secondary structure which lies between the last residue of the α-helix in a zinc finger and the first residue of the β-sheet in the next zinc finger. The linker sequence therefore joins together two zinc fingers. Typically, the last amino acid in a zinc finger is a threonine residue, which caps the α-helix of the zinc finger, while a tyrosine/phenylalanine or another hydrophobic residue is the first amino acid of the following zinc finger. Accordingly, in a “wild type” zinc finger, glycine is the first residue in the linker, and proline is the last residue of the linker. Thus, for example, in the Zif)₆₈ construct, the linker sequence is G(E/Q)(K/R)P.

A “flexible” linker is an amino acid sequence which does not have a fixed structure (secondary or tertiary structure) in solution. Such a flexible linker is therefore free to adopt a variety of conformations. An example of a flexible linker is the canonical linker sequence GERP/GEKP/GQRP/GQKP. Flexible linkers are also disclosed in WO99/45132 (Kim and Pabo). By “structured linker” we mean an amino acid sequence which adopts a relatively well-defined conformation when in solution. Structured linkers are therefore those which have a particular secondary and/or tertiary structure in solution.

Determination of whether a particular sequence adopts a structure may be done in various ways, for example, by sequence analysis to identify residues likely to participate in protein folding, by comparison to amino acid sequences which are known to adopt certain conformations (e.g., known alpha-helix, beta-sheet or zinc finger sequences), by NMR spectroscopy, by X-ray diffraction of crystallised peptide containing the sequence, etc as known in the art.

The structured linkers of our invention preferably do not bind nucleic acid, but where they do, then such binding is not sequence specific. Binding specificity may be assayed for example by gel-shift as described below.

The linker may comprise any amino acid sequence that does not substantially hinder interaction of the nucleic acid binding modules with their respective target subsites. Preferred amino acid residues for flexible linker sequences include, but are not limited to, glycine, alanine, serine, threonine proline, lysine, arginine, glutamine and glutamic acid.

The linker sequences between the nucleic acid binding domains preferably comprise five or more amino acid residues. The flexible linker sequences according to our invention consist of 5 or more residues, preferably, 5, 6, 7, 8, 9, 10, 11, 12, 13, 14, 15, 16, 17, 18, 19 or 20 or more residues. In a highly preferred embodiment of the invention, the flexible linker sequences consist of 5, 7 or 10 residues.

Once the length of the amino acid sequence has been selected, the sequence of the linker may be selected, for example by phage display technology (see for example U.S. Pat. No. 5,260,203) or using naturally occurring or synthetic linker sequences as a scaffold (for example, GQKP and GEKP, see Liu et al., 1997, Proc. Natl. Acad. Sci. USA 94, 5525-5530 and Whitlow et al., 1991, Methods: A Companion to Methods in Enzymology 2: 97-105). The linker sequence may be provided by insertion of one or more amino acid residues into an existing linker sequence of the nucleic acid binding polypeptide. The inserted residues may include glycine and/or serine residues. Preferably, the existing linker sequence is a canonical linker sequence selected from GEKP, GERP, GQKP and GQRP. More preferably, each of the linker sequences comprises a sequence selected from GGEKP, GGQKP, GGSGEKP, GGSGQKP, GGSGGSGEKP, and GGSGGSGQKP.

Structured linker sequences are typically of a size sufficient to confer secondary or tertiary structure to the linker; such linkers may be up to 30, 40 or 50 amino acids long. In a preferred embodiment, the structured linkers are derived from known zinc fingers which do not bind nucleic acid, or are not capable of binding nucleic acid specifically. An example of a structured linker of the first type is TFIIIA finger IV; the crystal structure of TFIIIA has been solved, and this shows that finger IV does not contact the nucleic acid (Nolte et al., 1998, Proc. Natl. Acad. Sci. USA 95, 2938-2943.). An example of the latter type of structured linker is a zinc finger which has been mutagenised at one or more of its base contacting residues to abolish its specific nucleic acid binding capability. Thus, for example, a ZIF finger 2 which has residues −1, 2, 3 and 6 of the recognition helix mutated to serines so that it no longer specifically binds DNA may be used as a structured linker to link two nucleic acid binding domains.

The use of structured or rigid linkers to jump the minor groove of DNA is likely to be especially beneficial in (i) linking zinc fingers that bind to widely separated (>3 bp) DNA sequences, and (ii) also in minimising the loss of binding energy due to entropic factors.

Typically, the linkers are made using recombinant nucleic acids encoding the linker and the nucleic acid binding modules, which are fused via the linker amino acid sequence. The linkers may also be made using peptide synthesis and then linked to the nucleic acid binding modules. Methods of manipulating nucleic acids and peptide synthesis methods are known in the art (see, for example, Maniatis, et al., 1991. Molecular Cloning: A Laboratory Manual. Cold Spring Harbor, N.Y., Cold Spring Harbor Laboratory Press).

Transcriptional Activators and Repressors

The nucleic acid binding polypeptides according to our invention may be linked to one or more transcriptional effector domains, such as an activation domain or a repressor domain.

In one embodiment of the invention, nucleic acid binding polypeptides comprising repressor domains are used to down-regulate expression of genes in primary cells. The repressor domain is preferably a transcriptional repressor domain selected from the group consisting of: a KRAB-A domain, an engrailed domain and a snag domain. Such a nucleic acid binding polypeptide may comprise nucleic acid binding domains linked by at least one flexible linker, one or more domains linked by at least one structured linker, or both.

Thus, a repressor of gene expression may be fused to the nucleic acid binding polypeptide and used to down regulate the expression of a gene contiguous or incorporating the nucleic acid binding polypeptide target sequence. Such repressors are known in the art and include, for example, the KRAB-A domain (Moosmann et al., Biol. Chem. 378: 669-677 (1997)), the KRAB domain from human KOX1 protein (Margolin et al., PNAS 91:4509-4513 (1994)). Molecules according to the invention comprising zinc finger proteins may be fused to transcriptional repression domains such as the Kruppel-associated box (KRAB) domain to form powerful repressors. These fusions are known to repress expression of a reporter gene even when bound to sites a few kilobase pairs upstream from the promoter of the gene (Margolin et al., 1994, PNAS USA 91, 4509-4513). Other repressor domains of use include the engrailed domain (Han et al., Embo J. 12: 2723-2733 (1993)) and the snag domain (Grimes et al., Mol Cell. Biol. 16: 6263-6272 (1996)). These can be used alone or in combination to down-regulate gene expression.

In another embodiment of the invention, nucleic acid binding polypeptides comprising activator domains are used to down-regulate expression of genes in primary cells. Examples of transcriptional activation domains include the VP16 and VP64 transactivation domains of Herpes Simplex Virus. Alternative transactivation domains are various and include the transactivation domain 1 and/or domain 2 of the p65 (RelA) subunit of nuclear factor-KB (NF-κB, Schrnitz, M. L. et al., J. Biol. Chem. 270: 15576-15584 (1995)), and the activation domain of CTCF (Vostrov, A. A. & Quitschke, W. W. J. Biol. Chem. 272: 33353-33359 (1997)). Other transcription activator domains which may be used include transcription factors reviewed in, for example, Lekstrom-Himes J. & Xanthopoulos K. G. (CIEBP family, J. Biol. Chem. 273: 28545-28548 (1998)), Bieker, J. J. et al., (globin gene transcription factors, Ann. N.Y. Acad. Sci. 850: 64-69 (1998), and Parker, M. G. (oestrogen receptors, Biochem. Soc. Symp. 63: 45-50 (1998)).

Use of a transactivation domain from the estrogen receptor is disclosed in Metivier, R., Petit, F G., Valotaire, Y. & Pakdel, F. (2000) Mol. Endocrinol. 14: 1849-1871. Furthermore, activation domains from the globin transcription factors EKLF (Pandya, K. Donze, D. & Townes T. (2001)J. Biol. Chem. 276: 8239-8243) may also be used, as well as a transactivation domain from FKLF (Asano, H. Li, X S. & Stamatoyannopoulos, G. (1999) Mol. Cell. Biol. 19: 3571-3579). C/EPB transactivation domains may also be employed in the methods described here. The C/EBP epsilon activation domain is disclosed in Verbeek, W., Gombart, A F, Chumakov, A M, Muller, C, Friedman, A D, & Koeffler, H P (1999) Blood 15: 3327-3337. Kowenz-Leutz, E. & Leutz, A. (1999) Mol. Cell. 4: 735-743 discloses the use of the CIEBP tao activation domain, while the C/EBP alpha transactivation domain is disclosed in Tao, H., & Umek, R M. (1999) DNA Cell Biol. 18: 75-84.

Variants and Derivatives

The nucleic acid binding polypeptide molecule as provided by the present invention includes splice variants encoded by mRNA generated by alternative splicing of a primary transcript, amino acid mutants, glycosylation variants and other covalent derivatives of said molecule which retain the physiological and/or physical properties of said molecule, such as its nucleic acid binding activity. Exemplary derivatives include molecules wherein the protein of the invention is covalently modified by substitution, chemical, enzymatic, or other appropriate means with a moiety other than a naturally occurring amino acid. Such a moiety may be a detectable moiety such as an enzyme or a radioisotope, or may be a molecule capable of facilitating crossing of cell membrane(s) etc.

Derivatives can be fragments of the nucleic acid binding molecule. Fragments of said molecule comprise individual domains thereof, as well as smaller polypeptides derived from the domains. Preferably, smaller polypeptides derived from the molecule according to the invention define a single epitope which is characteristic of said molecule. Fragments may in theory be almost any size, as long as they retain one characteristic of the nucleic acid binding molecule. Preferably, fragments may be at least 3 amino acids and in length.

Derivatives of the nucleic acid binding molecule also comprise mutants thereof, which may contain amino acid deletions, additions or substitutions, subject to the requirement to maintain at least one feature characteristic of said molecule. Thus, conservative amino acid substitutions may be made substantially without altering the nature of the molecule, as may truncations from the N- or C-terminal ends, or the corresponding 5′- or 3′-ends of a nucleic acid encoding it. Deletions or substitutions may moreover be made to the fragments of the molecule comprised by the invention. Nucleic acid binding molecule mutants may be produced from a DNA encoding a nucleic acid binding protein which has been subjected to in vitro mutagenesis resulting e.g. in an addition, exchange and/or deletion of one or more amino acids. For example, substitutional, deletional or insertional variants of the molecule can be prepared by recombinant methods and screened for nucleic acid binding activity as described herein.

The fragments, mutants and other derivatives of the polypeptide nucleic acid binding molecule preferably retain substantial homology with said molecule. As used herein, “homology” means that the two entities share sufficient characteristics for the skilled person to determine that they are similar in origin and/or function. Preferably, homology is used to refer to sequence identity. Thus, the derivatives of the molecule preferably retain substantial sequence identity with the sequence of said molecule.

“Substantial homology”, where homology indicates sequence identity, means more than 75% sequence identity and most preferably a sequence identity of 90% or more. Amino acid sequence identity may be assessed by any suitable means, including the BLAST comparison technique which is well known in the art, and is described in Ausubel et al., Short Protocols in Molecular Biology (1999) 4^{th} Ed, John Wiley & Sons, Inc.

Mutations

Mutations may be performed by any method known to those of skill in the art. Preferred, however, is site-directed mutagenesis of a nucleic acid sequence encoding the protein of interest. A number of methods for site-directed mutagenesis are known in the art, from methods employing single-stranded phage such as M13 to PCR-based techniques (see “PCR Protocols: A guide to methods and applications”, M. A. Innis, D. H. Gelfand, J. J. Sninsky, T. J. White (eds.). Academic Press, New York, 1990). Preferably, the commercially available Altered Site II Mutagenesis System (Promega) may be employed, according to the directions given by the manufacturer.

Screening of the proteins produced by mutant genes is preferably performed by expressing the genes and assaying the binding ability of the protein product. A simple and advantageously rapid method by which this may be accomplished is by phage display, in which the mutant polypeptides are expressed as fusion proteins with the coat proteins of filamentous bacteriophage, such as the minor coat protein pII of bacteriophage m13 or gene III of bacteriophage Fd, and displayed on the capsid of bacteriophage transformed with the mutant genes. The target nucleic acid sequence is used as a probe to bind directly to the protein on the phage surface and select the phage possessing advantageous mutants, by affinity purification. The phage are then amplified by passage through a bacterial host, and subjected to further rounds of selection and amplification in order to enrich the mutant pool for the desired phage and eventually isolate the preferred clone(s). Detailed methodology for phage display is known in the art and set forth, for example, in U.S. Pat. No. 5,223,409; Choo and Klug, (1995) Current Opinions in Biotechnology 6:431-436; Smith, (1985) Science 228:1315-1317; and McCafferty et al., (1990) Nature 348:552-554; all incorporated herein by reference. Vector systems and kits for phage display are available commercially, for example from Pharmacia.

The present invention allows the production of what are essentially artificial nucleic acid binding proteins. In these proteins, artificial analogues of amino acids may be used, to impart the proteins with desired properties or for other reasons. Thus, the term “amino acid”, particularly in the context where “any amino acid” is referred to, means any sort of natural or artificial amino acid or amino acid analogue that may be employed in protein construction according to methods known in the art. Moreover, any specific amino acid referred to herein may be replaced by a functional analogue thereof, particularly an artificial functional analogue. The nomenclature used herein therefore specifically comprises within its scope functional analogues of the defined amino acids.

The polypeptides which comprise the libraries according to the invention may comprise zinc finger polypeptides. In other words, they comprise a Cys2-His2 zinc finger motif.

Molecules according to the invention may advantageously comprise multiple zinc finger motifs. For example, molecules according to the invention may comprise any number of motifs, such as three zinc finger motifs, or may comprise four or five such motifs, or may comprise six zinc finger motifs, or even more. Advantageously, molecules according to the invention may comprise zinc finger motifs in multiples of three, such as three, six, nine or even more zinc finger motifs. Preferably, molecules according to the invention may comprise about three to about six zinc finger motifs.

Vectors

The nucleic acid encoding the nucleic acid binding protein for use in regulating gene expression in primary cells may be incorporated into vectors for further manipulation, or for purposes of constructing an expression construct suitable for introduction into a primary cell.

As used herein, vector (or plasmid) refers to discrete elements that are used to introduce heterologous nucleic acid into cells for either expression or replication thereof. Selection and use of such vehicles are well within the skill of the person of ordinary skill in the art. Many vectors are available, and selection of appropriate vector will depend on the intended use of the vector, i.e. whether it is to be used for DNA amplification or for nucleic acid expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2 μ plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. DNA may also be replicated by insertion into the host genome. However, the recovery of genomic DNA encoding the nucleic acid binding protein is more complex than that of exogenously replicated vector because restriction enzyme digestion is required to excise nucleic acid binding protein DNA. DNA can be amplified by PCR and be directly transfected into the host cells without any replication component.

Advantageously, an expression or cloning vector as described above may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Since the replication of vectors is conveniently done in E. coli, an E. coli genetic marker and an E. coli origin of replication are advantageously included. These can be obtained from E. coli plasmids, such as pBR322, Bluescript® vector or a pUC plasmid, e.g. pUC18 or pUC19, which contain both E. Coli replication origin and E. coli genetic marker conferring resistance to antibiotics, such as ampicillin.

Suitable selectable markers for mammalian cells are those that enable the identification of cells competent to take up nucleic acid binding protein nucleic acid, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked DNA that encodes the nucleic acid binding protein. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression

Expression and cloning vectors usually contain a promoter that is recognised by the host organism and is operably linked to nucleic acid binding protein encoding nucleic acid. Such a promoter may be inducible or constitutive. The promoters are operably linked to DNA encoding the nucleic acid binding protein by removing the promoter from the source DNA by restriction enzyme digestion and inserting the isolated promoter sequence into the vector. Both the native nucleic acid binding protein promoter sequence and many heterologous promoters may be used to direct amplification and/or expression of nucleic acid binding protein encoding DNA.

Promoters suitable for use with prokaryotic hosts include, for example, the β-lactamase and lactose promoter systems, alkaline phosphatase, the tryptophan (Trp) promoter system and hybrid promoters such as the tac promoter. Their nucleotide sequences have been published, thereby enabling the skilled worker operably to ligate them to DNA encoding nucleic acid binding protein, using linkers or adapters to supply any required restriction sites. Promoters for use in bacterial systems will also generally contain a Shine-Delgarno sequence operably linked to the DNA encoding the nucleic acid binding protein.

Preferred expression vectors are bacterial expression vectors which comprise a promoter of a bacteriophage such as phagex or T7 which is capable of functioning in the bacteria. In one of the most widely used expression systems, the nucleic acid encoding the fusion protein may be transcribed from the vector by T7 RNA polymerase (Studier et al, Methods in Enzymol. 185; 60-89, 1990). In the E. Coli BL21(DE3) host strain, used in conjunction with pET vectors, the T7 RNA polymerase is produced from the λ-lysogen DE3 in the host bacterium, and its expression is under the control of the IPTG inducible lac Uv5 promoter. This system has been employed successfully for over-production of many proteins. Alternatively the polymerase gene may be introduced on a lambda phage by infection with an int-phage such as the CE6 phage which is commercially available (Novagen, Madison, USA). other vectors include vectors containing the lambda PL promoter such as PLEX (Invitrogen, NL), vectors containing the trc promoters such as pTrcH is Xpress™ (Invitrogen) or pTrc99 (Pharmacia Biotech, SE) or vectors containing the tac promoter such as pKK223-3 (Pharm-acia Biotech) or PMAL (New England Biolabs, MA, USA).

Moreover, the nucleic acid binding protein gene according to the invention preferably includes a secretion sequence in order to facilitate secretion of the polypeptide from bacterial hosts, such that it will be produced as a soluble native peptide rather than in an inclusion body. The peptide may be recovered from the bacterial periplasmic space, or the culture medium, as appropriate. A “leader” peptide may be added to the N-terminal finger. Preferably, the leader peptide is MAEEKP.

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3-phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PHO5 promoter is e.g. a shortened acid phosphatase PH05 promoter devoid of the upstream regulatory elements (UAS) such as the PH05 (−173) promoter element starting at nucleotide −173 and ending at nucleotide −9 of the PH05 gene.

Nucleic acid binding protein gene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, and from the promoter normally associated with nucleic acid binding protein sequence, provided such promoters are compatible with the host cell systems.

Transcription of a DNA encoding nucleic acid binding protein by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to nucleic acid binding protein DNA, but is preferably located at a site 5′ from the promoter.

Advantageously, a eukaryotic expression vector encoding a nucleic acid binding protein according to the invention may comprise a locus control region (LCR). LCRs are capable of directing high-level integration site independent expression of transgenes integrated into host cell chromatin, which is of importance especially where the nucleic acid binding protein gene is to be expressed in the context of a permanently-transfected eukaryotic cell line in which chromosomal integration of the vector has occurred, or in transgenic animals.

Eukaryotic vectors may also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding nucleic acid binding protein.

An expression vector includes any vector capable of expressing nucleic acid binding protein nucleic acids that are operatively linked with regulatory sequences, such as promoter regions, that are capable of expression of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, DNAs encoding nucleic acid binding protein may be inserted into a vector suitable for expression of cDNAs in mammalian cells, e.g. a CMV enhancer-based vector such as pEVRF (Matthias, et al., (1989) NAR 17, 6418).

Particularly useful for practising the present invention are expression vectors that provide for the transient expression of DNA encoding nucleic acid binding protein in mammalian cells. Transient expression usually involves the use of an expression vector that is able to replicate efficiently in a host cell, such that the host cell accumulates many copies of the expression vector, and, in turn, synthesises high levels of nucleic acid binding protein. For the purposes of the present invention, transient expression systems are useful e.g. for identifying nucleic acid binding protein mutants, to identify potential phosphorylation sites, or to characterise functional domains of the protein.

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing nucleic acid binding protein expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

In accordance with another embodiment of the present invention, there are provided cells containing the above-described nucleic acids. Such host cells such as prokaryote, yeast and higher eukaryote cells may be used for replicating DNA and producing the nucleic acid binding protein. Suitable prokaryotes include eubacteria, such as Grain-negative or Gran-positive organisms, such as E. coli, e.g. E. coli K-12 strains, DH5a and HB101, or Bacilli. Further hosts suitable for the nucleic acid binding protein encoding vectors include eukaryotic microbes such as filamentous fungi or yeast, e.g. Saccharomyces cerevisiae. Higher eukaryotic cells include insect and vertebrate cells, particularly mammalian cells including human cells or nucleated cells from other multicellular organisms. In recent years propagation of vertebrate cells in culture (tissue culture) has become a routine procedure. Examples of useful mammalian host cell lines are epithelial or fibroblastic cell lines such as Chinese hamster ovary (CHO) cells, NIH 3T3 cells, HeLa cells or 293T cells. The host cells referred to in this disclosure comprise cells in in vitro culture as well as cells that are within a host animal.

DNA may be stably incorporated into cells or may be transiently expressed using methods known in the art. Stably transfected mammalian cells may be prepared by transfecting cells with an expression vector having a selectable marker gene, and growing the transfected cells under conditions selective for cells expressing the marker gene. To prepare transient transfectants, mammalian cells are transfected with a reporter gene to monitor transfection efficiency.

To produce such stably or transiently transfected cells, the cells should be transfected with a sufficient amount of the nucleic acid binding protein-encoding nucleic acid to form the nucleic acid binding protein. The precise amounts of DNA encoding the nucleic acid binding protein may be empirically determined and optimised for a particular cell and assay.

Host cells are transfected-or, preferably, transformed with the above-captioned expression or cloning vectors of this invention and cultured in conventional nutrient media modified as appropriate for inducing promoters, selecting transformants, or amplifying the genes encoding the desired sequences. Heterologous DNA may be introduced into host cells by any method known in the art, such as transfection with a vector encoding a heterologous DNA by the calcium phosphate coprecipitation technique or by electroporation. Numerous methods of transfection are known to the skilled worker in the field. Successful transfection is generally recognised when any indication of the operation of this vector occurs in the host cell. Transformation is achieved using standard techniques appropriate to the particular host cells used.

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (see, e.g. Sambrook et al. (1989) Molecular Cloning: A Laboratory Manual, Second Edition, Cold Spring Harbor Laboratory Press).

Transfected or transformed cells are cultured using media and culturing methods known in the art, preferably under conditions, whereby the nucleic acid binding protein encoded by the DNA is expressed. The composition of suitable media is known to those in the art, so that they can be readily prepared. Suitable culturing media are also commercially available.

Target Genes and Nucleotide Sequences

The term “target gene” refers to a gene or other coding sequence, the expression of which can be affected using compositions and methods described in the present invention. A target gene may be an endogenous gene (i.e. one which is normally found in the genome of the animal or animal cell) or a heterologous gene (i.e. one that does not normally exist in the genome of the animal or cell).

Genes that provide suitable targets for the nucleic acid binding polypeptides of our invention include those involved in diseases such as cardiovascular (low-density lipoprotein receptor, CDH1, ABC1, apolipoproteinA-I, ApoA-II, ApoA-IV, ApoE, lipoprotein lipase, LCAT, SR-BI, CETP etc), inflammatory (IL-1β, IL-1 Ra, IL-4, IL-10, IL-13, TNF-α etc), metabolic, infectious (viral, bacteria, fungal, etc), genetic, neurological, rheumatological, dermatological, and musculoskeletal diseases.

Also those genes involved in biochemical pathways that synthesise biologically useful (casein), or unwanted products (lactose) in animal products for human consumption, or those involved in the production of valuable therapeutic (factor VIII, factor IX, IGF-1, insulin, antibodies) or industrial products, and those involved in immune rejection of xenotransplants (porcine alpha-1,3-galactosyltransferase), for the creation of useful transgenic animals (see First, N. L. & Thomson, J. Nat. Biotechnol. 16: 620-621 (1998); Colman, A. Biochem. Soc. Symp. 63: 141-147 (1998); Pennisi, E. Science 279: 646-648 (1998); Whitelaw, B. Nat. Biotechnol. 17: 135-136 (1999); Brink M. F. et al., Theriogenology 53: 139-148 (2000); Smith L. C. et al., Can. Vet. J. 41: 919-924 (2000) and Wolf, E. et al., Exp. Physiol. 85: 615-625 (2000) for reviews).

In particular, the invention provides nucleic acid binding peptides suitable for the treatment of diseases, syndromes and conditions such as hypertrophic cardiomyopathy, bacterial endocarditis, agyria, amyotrophic lateral sclerosis, tetralogy of fallot, myocarditis, anemia, brachial plexus, neuropathies, hemorrhoids, congenital heart defects, alopecia areata, sickle cell anemia, mitral valve prolapse, autonomic nervous system diseases, alzheimer disease, angina pectoris, rectal diseases, arrhythmogenic right, ventricular dysplasia, acne rosacea, amblyopia, ankylosing spondylitis, atrial fibrillation, cardiac tamponade, acquired immunodeficiency syndrome, amyloidosis, autism, brain neoplasms, central nervous system diseases, colour vision defects, arteriosclerosis, breast diseases, central nervous system infections, colorectal neoplasms, arthritis, behcet's syndrome, breast neoplasms, cerebral palsy, common cold, asthma, bipolar disorder, burns, cervix neoplasms, communication disorders, atherosclerosis, candidiasis, charcot-marie disease, crohn disease, attention deficit disorder, brain injuries, cataract, ulcerative colitis, cumulative trauma disorders, cystic fibrosis, developmental disabilities, eating disorders, erysipelas, fibromyalgia, decubitus ulcer, diabetes, emphysema, escherichia coli infections, folliculitis, deglutition disorders, diabetic foot, encephalitis, oesophageal diseases, food hypersensitivity, dementia, down syndrome, japanese encephalitis, eye neoplasms, dengue, dyslexia, endometriosis, fabry's disease, gastroenteritis, depression, dystonia, chronic fatigue syndrome, gastroesophageal reflux, gaucher's disease, hematologic diseases, hirschsprung disease, hydrocephalus, hyperthyroidism, gingivitis, hemophilia, histiocytosis, hyperhidrosis, hypoglycemia, glaucoma, hepatitis, hiv infections, hyperoxaluria, hypothyroidism, glycogen storage disease, hepatolenticular degeneration, hodgkin disease, hypersensitivity, immunologic deficiency syndromes, hernia, holt-oram syndrome, hypertension, impotence, congestive heart failure, herpes genitalis, huntington's disease, pulmonary hypertension, incontinence, infertility, leukemia, systemic lupus erythematosus, maduromycosis, mental retardation, inflammation, liver neoplasms, lyme disease, malaria, inborn errors of metabolism, inflammatory bowel diseases, long qt syndrome, lymphangiomyomatosis, measles, migraine, influenza, low back pain, lymphedema, melanoma, mouth abnormalities, obstructive lung diseases, lymphoma, meningitis, mucopolysaccharidoses, leprosy, lung neoplasms, macular degeneration, menopause, multiple sclerosis, muscular dystrophy, myofascial pain syndromes, osteoarthritis, pancreatic neoplasms, peptic ulcer, myasthenia gravis, nausea, osteoporosis, panic disorder, myeloma, acoustic neuroma, otitis media, paraplegia, phenylketonuria, myeloproliferative disorders, nystagmus, ovarian neoplasms, parkinson disease, pheochromocytoma, myocardial diseases, opportunistic infections, pain, pars planitis, phobic disorders, myocardial infarction, hereditary optic atrophy, pancreatic diseases, pediculosis, plague, poison ivy dermatitis, prion diseases, reflex sympathetic dystrophy, schizophrenia, shyness, poliomyelitis, prostatic diseases, respiratory tract diseases, scleroderma, sjogren's syndrome, polymyalgia rheumatica, prostatic neoplasms, restless legs, scoliosis, skin diseases, postpoliomyelitis syndrome, psoriasis, retinal diseases, scurvy, skin neoplasms, precancerous conditions, rabies, retinoblastoma, sex disorders, sleep disorders, pregnancy, sarcoidosis, sexually transmitted diseases, spasmodic torticollis, spinal cord injuries, testicular neoplasms, trichotillomania, urinary tract, infections, spinal dystaphism, substance-related disorders, thalassemia, trigeminal neuralgia, urogenital diseases, spinocerebellar degeneration, sudden infant death, thrombosis, tuberculosis, vascular diseases, strabismus, tinnitus, tuberous sclerosis, post-traumatic stress disorders, syringomyelia, tourette syndrome, tumer's syndrome, vision disorders, psychological stress, temporomandibular joint dysfunction syndrome, trachoma, urinary incontinence, von willebrand's disease, renal osteodystrophy, bacterial infections, digestive system neoplasms, bone neoplasms, vulvar diseases, ectopic pregnancy, tick-borne diseases, marfan syndrome, aging, williams syndrome, angiogenesis factor, urticaria, sepsis, malabsorption syndromes, wounds and injuries, cerebrovascular accident, multiple chemical sensitivity, dizziness, hydronephrosis, yellow fever, neurogenic arthropathy, hepatocellular carcinoma, pleomorphic adenoma, vater's ampulla, meckel's diverticulum, keratoconus skin, warts, sick building syndrome, urologic diseases, ischemic optic neuropathy, common bile duct calculi, otorhinolaryngologic diseases, superior vena cava syndrome, sinusitis, radius fractures, osteitis deformans, trophoblastic neoplasms, chondrosarcoma, carotid stenosis, varicose veins, creutzfeldt-jakob syndrome, gallbladder diseases, replacement of joint, vitiligo, nose diseases, environmental illness, megacolon, pneumonia, vestibular diseases, cryptococcosis, herpes zoster, fallopian tube neoplasms, infection, arrhythmia, glucose intolerance, neuroendocrine tumors, scabies, alcoholic hepatitis, parasitic diseases, salpingitis, cryptococcal meningitis, intracranial aneurysm, calculi, pigmented nevus, rectal neoplasms, mycoses, hemangioma, colonic neoplasms, hypervitaminosis a, nephrocalcinosis, kidney neoplasms, vitamins, carcinoid tumor, celiac disease, pituitary diseases, brain death, biliary tract diseases, prostatitis, iatrogenic disease, gastrointestinal hemorrhage, adenocarcinoma, toxic megacolon, amputees, seborrheic keratosis, osteomyelitis, barrett esophagus, hemorrhage, stomach neoplasms, chickenpox, cholecystitis, chondroma, bacterial infections and mycoses, parathyroid neoplasms, spermatic-cord torsion, adenoma, lichen planus, anal gland neoplasms, lipoma, tinea pedis, alcoholic liver diseases, neurofibromatoses, lymphatic diseases, elder abuse, eczema, diverticulitis, carcinoma, pancreatitis, amebiasis, pyelonephritis, and infectious mononucleosis, etc.

Most commonly, target nucleotide sequences will be sequences associated with a target gene that is to be regulated by a nucleic acid binding polypeptide. The term “target nucleotide sequence” means any nucleic acid sequence to which a nucleic acid binding polypeptide such as a zinc finger peptide is capable of binding. It is usually a DNA sequence within an animal chromosome (but may be an RNA transcript), to which a nucleic acid binding polypeptide is capable of binding. A target DNA sequence will generally be associated with a target gene (see above) and the binding of the nucleic acid binding polypeptide (e.g., a zinc finger polypeptide) to the DNA sequence will generally allow the up- or down-regulation of the associated coding sequence. Target nucleotide sequences include sequences which are naturally associated with target genes, their RNA transcripts, and also other sequences which can be configured with a target gene to allow the up- or down-regulation of such gene. For example, the known binding site of a given nucleic acid binding polypeptide may be a target DNA sequence and, when operably linked to a target gene, will allow expression of the target gene to be regulated by the given zinc finger protein. Similarly, the target nucleotide sequence may be an RNA sequence within the RNA transcript of the target gene. In this case, binding of the zinc finger peptide to the RNA will allow the half-life or targeting of the RNA to be controlled, leading to more or less expression of the associated gene.

Gene Therapy

The methods described here for targeting nucleic acid sequences in primary cells, and the nucleic acid binding polypeptides disclosed here which are capable of such binding and regulation, may be used for the purposes of gene therapy. Such gene therapy may be employed for prevention or treatment of diseases, conditions, syndromes, or the prevention or relief of any of their symptoms. Any of the nucleic acid binding polypeptides such as zinc fingers disclosed here may therefore be introduced into suitable target primary cell for such gene therapy.

As applied to the methods described here, gene therapy by targeting primary cells, as described above, is usefully employed as ex-vivo somatic cell therapy. Thus, cells are removed from the body of a patient and cultured as primary cells. Nucleic acid binding polypeptide is then introduced into the primary cells by for example transfection of a suitable construct, to regulate expression of the gene of interest. The primary cells may then be re-introduced into an organism, which may be the same organism from which the primary cells are derived.

For example, many symptoms associated with kidney failure are frequently due to anaemia and are refractory to kidney dialysis. Anaemia leaves dialysis patients fatigued and exhausted, impairing their ability to work or perform even routine tasks. This is caused by insufficient production of erythropoietin (EPO), a protein naturally produced by functioning kidneys, which circulates through the bloodstream to the bonemarrow, stimulating the production of red blood cells. Administration of recombinant EPO increases the haematocrit of sufferers and restores their ability to lead a normal life. Therefore, and as described in the Examples below, zinc finger polypeptides may be designed to target the erythropoietin promoter to promote expression of the erythropoietin protein (EPO). In particular, cells which do not normally produce EPO, such as human dermal fibroblasts, may be targeted to achieve expression and secretion of EPO, thereby recovering the normal balance of EPO in the blood stream in anaemic patients. Thus, human dermal fibroblasts may be taken from the body of a patient, cultured, and transfected with a zinc finger construct capable of upregulating expression of EPO. They may then be re-introduced into the patient so that EPO is secreted into the bloodstream.

Furthermore, primary pancreatic islet cells may be targeted to promote expression of insulin to treat diabetes. Up-regulation of genes encoding autoantigens may be used to induce immunological tolerance and therefore to treat a variety of auto-immune diseases. Expression of oncogenes may be regulated in any sort of tumour cell (as primary cells) to treat or prevent cancer. Likewise, tumour suppressor genes such as p53 and Rb may be up-regulated.

Accordingly, the zinc finger polypeptides of the present invention may be introduced into cells as a means of preventing or treating diseases such as kidney failure as well as other diseases.

The target cell for introduction of the zinc finger will be chosen according to the condition or disease to be treated or prevented. The choice of suitable target primary cells will be known in the art. For example, for the treatment or prevention of skin diseases, the optimal target cell population for such strategy may comprise epidermal cells. Similarly, primary liver cells may be used as target cells for treatment or prevention of liver disease.

Zinc finger constructs may be introduced into the target cell by any suitable means, for example as nucleic acid based expression constructs. Plasmid and other expression constructs are described in detail elsewhere in this document. Virus based vectors (for example, viral expression constructs) may also be used advantageously to effect gene delivery into a target cell. The viral vector is essentially an engineered virus, and retains its ability to express the gene of interest as well as maintaining its ability to deliver this gene to target cells. Other expression vectors are known in the art, and may also be used. Thus, any suitable vector, preferably a viral based vector, may be used as a means of introducing the nucleic acid binding polypeptides of the invention into target cells.

Retroviral (oncoretrovirus or lentivirus) based vectors are particularly attractive for gene delivery as they integrate efficiently into the host chromosomal DNA, resulting in the stable transmission and expression of the transgene. Successful gene transfer into peripheral blood lymphocytes (PBLs) may be achieved with conventional oncoretroviral vectors, for example, those based on the Moloney murine leukemia virus (MoMuLV). Efficient retroviral gene transfer with MoMuLV-based vector to T cells may be achieved by using cytokine or/and antibody prestimulation, high titer pseudotyped retroviral vectors and co-localisation of retroviral particles and target cells.

The vector which may be used may include vectors, for example, based on the LNL (Bender, M. A., Palmer, T. D., Gelinas, R. E. & Miller, A. D. (1987) J. Virol. 61:1639-1646) or derivative MoMuLV-based oncoretroviral vector encoding a nucleic acid binding polypeptide gene. Alternatively a lentiviral or other vector could be used. Recombinant viral particles may be pseudotyped with amphotropic, feline endogenous retrovirus (RD114) envelope protein, Gibbon Ape Leukemia virus (GALV) envelope protein G protein of vesicular stomatitis virus (VSV-G) for successful infection of human cells. Other methods of gene introduction are discussed elsewhere in this document.

Gene therapy clinical protocols used for successful transduction are described in, for example, Conneally et al., 1998, Blood, 91, 3487-3493; Marandin et al., 1998, Hum Gene Ther, 9, 1497-1511; Schilz et al., 1998, Blood, 92, 3163-3171; van Hennik et al., 1998, Blood, 92, 40134022; Demaison et al., 2000, Hum Gene Ther, 11, 91-100; Guenechea et al., 2000, Mol Ther, 6, 566-573; Dardalon et al., 2000, Blood, 96, 885-893; Miyoshi et al., 1999, Science, 283, 682-686; Cavazzana-Calvo et al., 2000, Science 288, 669-672; Abonour et al., 2000, Nature Medicine, 6, 652-658.

Pharmaceuticals

Primary cells which have been manipulated by the methods described here to regulate gene expression may be introduced into an organism for treatment. For example, primary cells may be transfected with constructs expressing a particular nucleic acid binding polypeptide (such as a zinc finger polypeptide), which is capable of targeting a nucleic acid sequence to up-regulate expression of a polypeptide of interest. Such primary cells may be administered to a patient in need of the polypeptide of interest. Primary cells are preferably administered in the form of pharmaceutical compositions.

The pharmaceutical preparations according to the invention which contain the primary cells are those for enteral, such as oral, furthermore rectal, and parenteral administration to (a) warm-blooded animal(s), the active ingredient being present on its own or together with a pharmaceutically acceptable carrier. The daily dose of the active ingredient depends on the age and the individual condition and also on the manner of administration.

The novel pharmaceutical preparations contain, for example, from about 10% to about 80%, preferably from about 20% to about 60%, of the active ingredient (i.e., primary cells). Pharmaceutical preparations according to the invention for enteral or parenteral administration are, for example, those in unit dose forms, such as sugar-coated tablets, tablets, capsules or suppositories, and furthermore ampoules. These are prepared in a manner known per se, for example by means of conventional mixing, granulating, sugar-coating, dissolving or lyophilising processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

Suitable carriers are, in particular, fillers, such as sugars, for example lactose, sucrose, mannitol or sorbitol, cellulose preparations and/or calcium phosphates, for example tricalcium phosphate or calcium hydrogen phosphate, furthermore binders, such as starch paste, using, for example, corn, wheat, rice or potato starch, gelatin, tragacanth, methylcellulose and/or polyvinylpyrrolidone, if desired, disintegrants, such as the abovementioned starches, furthermore carboxymethyl starch, crosslinked polyvinylpyrrolidone, agar, alginic acid or a salt thereof, such as sodium alginate; auxiliaries are primarily glidants, flow-regulators and lubricants, for example silicic acid, talc, stearic acid or salts thereof, such as magnesium or calcium stearate, and/or polyethylene glycol. Sugar-coated tablet cores are provided with suitable coatings which, if desired, are resistant to gastric juice, using, inter alia, concentrated sugar solutions which, if desired, contain gum arabic, talc, polyvinylpyrrolidone, polyethylene glycol and/or titanium dioxide, coating solutions in suitable organic solvents or solvent mixtures or, for the preparation of gastric juice-resistant coatings, solutions of suitable cellulose preparations, such as acetylcellulose phthalate or hydroxypropylmethylcellulose phthalate. Colorants or pigments, for example to identify or to indicate different doses of active ingredient, may be added to the tablets or sugar-coated tablet coatings.

Other orally utilisable pharmaceutical preparations are hard gelatin capsules, and also soft closed capsules made of gelatin and a plasticiser, such as glycerol or sorbitol. The hard gelatin capsules may contain the active ingredient in the form of granules, for example in a mixture with fillers, such as lactose, binders, such as starches, and/or lubricants, such as talc or magnesium stearate, and, if desired, stabilisers. In soft capsules, the active ingredient is preferably dissolved or suspended in suitable liquids, such as fatty oils, paraffin oil or liquid polyethylene glycols, it also being possible to add stabilisers.

Suitable rectally utilisable pharmaceutical preparations are, for example, suppositories, which consist of a combination of the active ingredient with a suppository base. Suitable suppository bases are, for example, natural or synthetic triglycerides, paraffin hydrocarbons, polyethylene glycols or higher alkanols. Furthermore, gelatin rectal capsules which contain a combination of the active ingredient with a base substance may also be used. Suitable base substances are, for example, liquid triglycerides, polyethylene glycols or paraffin hydrocarbons.

Suitable preparations for parenteral administration are primarily aqueous solutions of an active ingredient in water-soluble form, for example a water-soluble salt, and furthermore suspensions of the active ingredient, such as appropriate oily injection suspensions, using suitable lipophilic solvents or vehicles, such as fatty oils, for example sesame oil, or synthetic fatty acid esters, for example ethyl oleate or triglycerides, or aqueous injection suspensions which contain viscosity-increasing substances, for example sodium carboxymethylcellulose, sorbitol and/or dextran, and, if necessary, also stabilisers.

### Example 1

### Zinc Finger Engineering Strategy (TNFR1 Receptor)

Strategy

Sequences within the TNFR1 promoter region which show homology between different mammalian species (such as human and mouse), and regions that code for putative transcription factors such as AP-2 are targeted (see, for example, Kemper, O. & Wallach, D. Gene 134: 209-216 (1993)). Zinc fingers are engineered to bind to the TNFR1 promoter using the ‘bipartite’ method described above and in WO98/53057. The bipartite method is based on a pair of pre-made zinc finger phage display libraries, which are used in parallel to select two DNA-binding domains that each recognise given 5 bp sequences, and whose products are recombined to produce a single protein that recognises a composite (9-10 bp) site of predefined sequence. Engineering using this system can be completed in less than two weeks and yields three-zinc finger polypeptide molecules that bind sequence-specifically to DNA with Kds in the nanomolar range. Having thus obtained three-zinc finger molecules, the genes for these peptides are linked together to make functional six-zinc finger proteins.

Targeted Site

The specific DNA sequence in the promoter region of the TNFR1 gene that is used as a target for engineering 3-finger proteins using the ‘bipartite’ protocol, is shown below. The 9-bp binding site is underlined.

| | |
|---|---|
| TNFR1-4-2 | |
| 5′GTCGGATTGGTGGG TTGGGGGCACAAGGCA-3′ | (TNFR1-4-2) |

TNFR1-4-2 therefore recognises underlined sites in GGATTGGTGGG TTGGGGGCACA.

TNFR1-4-2 Zinc Finger Sequence

The amino acid sequence of the helical regions from the recombinant six-zinc finger DNA-binding domain (TNFR1-4-2) engineered against the TNFR1 gene promoter is shown below. Residues are numbered relative to the first position in the α-helix (position 1) in each finger (F1-6).

| F1 | F2 | F3 | F4 | F5 | F6 |
|---|---|---|---|---|---|
| −1123456 | −1123456 | −1123456 | −1123456 | −1123456 | −1123456 |
| ASADLTR | RRDHLSE | RNDSRTN | RSQHLTE | TSSHLSV | HSNARKT |

Amino acid linker TGSERP is used to link the three-finger units between F3 and F4 into six-finger constructs. The entire TNFR1-4-2 amino acid sequence, recognising GGATTGGTGGG TTGGGGGCACA, is shown below:

| | TNFR1-4-2 |
|---|---|
| 1 | MAERPYACPVESCDR |
| 16 | RFSASADLTRHIRIH |
| 31 | TGQKPFQCRICMRNF |
| 46 | SRRDHLSEHIRTHTG |
| 61 | EKPFACDICGRKFAR |
| 76 | NDSRTNHTKIHTGSE |
| 91 | RPYACPVESCDRRFS |
| 106 | RSQHLTEHIRIHTGQ |
| 121 | KPFQCRICMRNFSTS |
| 136 | SHLSVHIRTHTGEKP |
| 151 | FACDICGRKFAHSNA |
| 166 | RKTHTKIHLRQKD |

TNFR1-4-2-Kox Zinc Finger Repressor

The zinc finger protein selected to bind to the TNFR1 promoter region is then engineered into a repressor polypeptide. These repressor contains the zinc finger DNA binding domain at the N-terminus fused in frame to the translation initiation sequence ATG. The 7 amino acid nuclear localisation sequence (NLS) of the wild-type Simian Virus 40 large-T antigen (Kalderon et al., Cell 39:499-509 (1984)) is fused to the C-terminus of the zinc finger sequence and the Kruppel-associated box (KRAB) repressor domain from human KOX1 protein (Margolin et al., PNAS 91:45094513 (1994)) is fused downstream of the NLS.

The sequence of the SV40-NLS-KOX1-c-myc repressor domain (NLS-KOX1-c-myc domain sequence) is as follows:

AARNSGPKKKRKVDGGGALSPQHSAVTQGSIIKNKEGMDAKSLTAWSRTLVTFKD
VFVDFTREEWKLLDTAQQIVYRNVMLENYKNLVSLGYQLTKPDVILRLEKGEEPW
LVEREIHQETHPDSETAFEIKSSVEQKLISEEDL

The KOX1 domain contains amino acids 1-97 from the human KOX1 protein (database accession code P21506) in addition to 23 amino acids which act as a linker. In addition, a 10 amino acid sequence from the c-myc protein (Evan et al., Mol. Cell. Biol. 5: 3610 (1985)) is introduced downstream of the KOX1 domain as a tag to facilitate expression studies of the fusion protein.

The sequence of the TNFR1-4-2-Kox zinc finger repression construct is as follows:

MAERPYACPVESCDRRFSASADLTRHIRIHTGQKPFQCRICMRNFSRRDHLSEHI
RTHTGEKPFACDICGRKFARNDSRTNHTKIHTGSERPYACPVESCDRRFSRSQHLTEHIR
IHTGQKPFQCRICMRNFSTSSHLSVHIRTHTGEKPFACDICGRKFAHSNARKTHTKIHLR
QKDAARNSGPKKKRKVDGGGALSPQHSAVTQGSIIKNKEGMDAKSLTAWSRTLVTFKDVF
VDFTREEWKLLDTAQQIVYRNVMLENYKNLVSLGYQLTKPDVILRLEKGEEPWLVEREIH
QETHPDSETAFEIKSSVEQKLISEEDL

Zinc finger constructs are then tested for specific target binding using a fluorescence ELISA, and for repression activity using FACS analysis.

### Example 2

### Assay for Binding Affinity Using in vitro Fluorescence ELISA

The binding properties of TNFR1-4-2 are assayed using an in vitro zinc finger fluorescence ELISA DNA-binding assay to assess whether the proteins bind specifically to their respective target sequences.

Preparation of Template

Zinc finger constructs are inserted into the protein expression vector pTracer (Invitrogen), downstream of the T7 RNA transcription promoter. Suitable templates for in vitro ELISA are created by PCR using the 5′ primer (GCAGAGCTCTCTGGCTAACTAGAG), which binds upstream of the T7 promoter and a 3′ primer, which binds to the 3′ end of the zinc finger construct and adds a sequence encoding for the HA-antibody epitope tag (YPYDVPDYA).

Zinc Finger Expression

In vitro transcription and translation are performed using the T7 TNT Quick Coupled Transcription/Translation System for PCR templates (Promega), according to the manufacturers instructions, except that the medium is supplemented with 500 μM ZnCl₂.

Fluorescence ELISA

DNA binding reactions contain the appropriate zinc finger peptide, biotinylated binding site (10 nM) and 5 μg competitor DNA (sonicated salmon sperm DNA), in a total volume of 50 μl, which contained: 1×PBS (pH 7.0), 1.25×10⁻³ U high affinity anti-HA-Peroxidase antibody (Boehringer Mannheim), 50 μM ZnCl₂, 0.01 mg/ml BSA, and 0.5% Tween 20. Incubations are performed at room temperature for 40 minutes. Black streptavidin-coated wells are blocked with 4% marvel for 1 hour. Binding reactions are added to the streptavidin-coated wells and incubated for a further 40 minutes at room temperature. Wells are washed 5 times in 100 μl wash buffer (1×PBS (pH 7.0), 50 M ZnCl₂, 0.01 mg/ml BSA, and 0.5% Tween 20), and finally 50 μl QuantaBlu peroxidase substrate solution (Pierce) is added to detect bound HA-tagged zinc finger peptide. ELISA signals are read in a SPECTRAmax GeminiXS spectrophotometer (Molecular Devices) and analysed using SOFTmax Pro 3.1.2 (Molecular Devices).

It is found that TNFR1-4-2 binds to its target sequence with high affinity and specificity.

### Example 3

### Delivery of Zinc Fingers to Primary Cells Using a Viral Vector

The oncoretroviral vector used contains the TNFR1-4-2-Kox1 gene (see above) and cis-acting viral sequences for gene expression and viral replication, such as the Long Terminal Repeat (LTR), the primer binding site, the attachment site and polypurine tract sequences and an extended packaging signal. However, it has been deleted of all viral protein coding sequences (e.g. gag, pol, env), so it is unable to replicate and produce functional viral capsid without the assistance of a helper cell line (also known as a packaging cell line), or co-transfected plasmids encoding these required proteins. This vector has been used in many gene therapy clinical trials and has shown no sign of toxicity either ex vivo or in patients who have been treated.

The TNFR1-4-2-Kox1 gene is sub-cloned from the plasmid pTracer —CMV/Bsd (Invitrogen) using the PmeI restriction enzyme, into an LNL-type vector (Bender, M. A., Palmer, T. D., Gelinas, R. E. & Miller, A. D. (1987) J. Virol. 61:1639-1646) inserted into a pUC backbone. In this vector, the expression of TNFR1-4-2-Kox1 is under the transcriptional control of the Moloney murine leukemia virus (Mo-MuLV) long terminal repeat (LTR) or other suitable LTR. The viral vector also encodes a marker protein, the green fluorescent protein (GFP). The expression of this marker gene is also driven by the viral LTR, a mechanism made possible by the insertion of an internal ribosomal entry site (IRES) sequence between both genes.

Since the viral vector contains deletions of several essential viral protein genes (e.g. gag, pol, env), it is unable to replicate and produce functional viral capsid without the assistance of a helper cell line (also known as a packaging cell line), or a co-transfected plasmid.

Viral supernatant is produced by transient transfection of 293T cells, as described in detail in the following Example. The helper functions are provided from two different constructs, one expressing Gag-Pol encoding the viral capsid, reverse transcriptase and integrase but lacking the encapsidation signal normally present in the Gag region, and another expressing the envelope. For successful infection of human cells, the envelope protein used is derived from the feline endogenous retrovirus (RD114) envelope protein but alternatively the Gibbon Ape Leukemia virus (GALV) envelope protein or the G protein of vesicular stomatitis virus (VSV-G) may be used.

### Example 4

### Oncoretroviral Vector Production

RD114 pseudotyped vectors are produced by transient transfection of three plasmids into 293T cells: the transfer vector plasmid (LNL-based; Bender, M. A., Palmer, T. D., Gelinas, R. E. & Miller, A. D. (1987) J. Virol. 61:1639-1646), pHIT60 (from Prof Mary Collins' lab, UCL, London, UK); a helper packaging plasmid encoding GAG and POL proteins of murine leukemia virus; and pRDF (from Prof Mary Collins' lab, UCL, London, UK) encoding for the feline endogenous retrovirus (RD114) envelope protein.

A total of 1.5×10⁷ 293T cells are seeded in one 150-cm² flask over-night prior to transfection. Cells are cultured at 37° C. in Dulbecco's modified Eagle medium (DMEM) with 10% fetal calf serum (FCS), and standard amounts of glutamine, penicillin and streptomycin, in a 5% CO₂ incubator. A total of 72 μg of plasmid DNA is used for the transfection of one flask: 12 μg of the envelope plasmid (pRDF), 24 μg of packaging plasmid (pHIT60), and 36 μg of transfer vector (pRetro) plasmid. These plasmids are pre-complexed with lipofectamine 2000 (Life Technology) in Optimem according to the manufacturer instructions. The DNA plus lipofectamine complexes are added to the cells at 90% confluence.

After 4 hours incubation at 37° C. in a 5% CO₂ incubator, the medium is replaced by fresh DMEM or alternatively RPMI 1640 medium supplemented with 10% FCS, and further incubated at 33° C. to increase the half-life of the recombinant virus.

At 36 hours and 60 hours post-transfection, the medium is harvested, cleared by low-speed centrifugation (800 rpm, 15 min), filtered through 0.45-μm-pore-size filters and used directly, or kept at −80° C. until required.

### Example 5

### Transduction of Human Cells

Human umbilical vein endothelial cells (HUVEC) are infected with the recombinant viral vector encoding the TNFR1-4-2-Kox1 gene, produced as described in the above Example. An empty viral vector which expresses just GFP is used as a control.

HUVEC cells (Clonetics) are maintained in media according to the recommendations of the supplier. For successful infection with the recombinant viral vector, cells are harvested using trypsin/EDTA and 5×10⁴ cells are plated into each well of a 6-well cell culture plate. After 24 hours, viral preparations are added at the appropriate amount (usually 0.5 ml to a well containing 2 ml of medium). Polybrene is added to a concentration of 8 μg/ml to promote infection. The cells are then maintained under standard growth conditions and analysed for EGFP expression by cytofluorimetry after 24-48 hours to determine transduction rates.

### Example 6

### Specific Down-regulation of Endogenous TNFR1 In Primary Cells (FACS Analysis)

HUVEC cells are known to express TNFR1 on their cell surface and so this cell line can be used to demonstrate the regulation of TNFR1 expression by a zinc finger designed to repress the expression of its gene. To do this, fluorescence cytometry is used to measure the amount of the TNFR1 protein on the surface of cells expressing the TNFR1-4-2-Kox1 protein, and on those containing a control plasmid which expresses just GFP. When required, GFP positive cells are isolated by fluorescence activated cell sorting (FACS).

Expression of the zinc finger protein TNFR1-4-2-Kox1 in transfected cells is monitored by the co-expression of the green fluorescent protein (GFP), which is expressed from the same mRNA transcript as the zinc finger peptide. Cell samples are analysed between 24 and 72 hours post viral infection, and sorted according to the level of GFP expressed. As a further control, untransfected HUVEC cells are also monitored.

The level of TNFR1 on the surface of the HUVEC cells is analysed using the following protocol.

Cells are harvested and pelleted at 1000 rpm for 5 minutes at room temperature. Pellets are resuspended in 50 μl ice-cold staining buffer (PBS, 0.5% BSA, 0.01% sodium azide) and incubated for 30 minutes on ice with saturating amounts of antibodies.

To assess for TNFR1 expression, cells are stained with a mouse-anti-human TNFR1 antibody (550514, Pharmingen, 1:25 dilution), which is then bound by a biotinylated anti-mouse IgG, Fab-specific (Sigma), and detected with streptavidin-Cychrome (Pharmingen). Between each step, cells are pelleted at 1000 rpm for 5 minutes and washed twice in 500 μl ice-cold staining buffer. Flow cytometric analysis is performed on a FACSCalibur using the CellQuest software package (Becton Dickinson). About 300,000 events corresponding to 15,000 events gated on GFP positive cells are collected per sample.

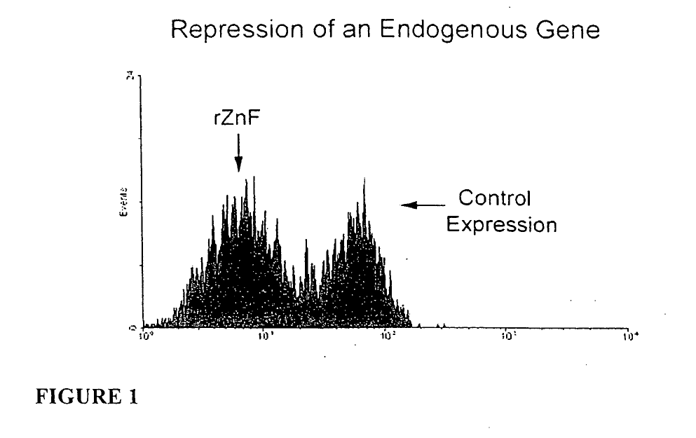

FIG. 1 demonstrates the results obtained 72 hours post viral infection. The graph shows the amount of TNFR1 protein on the surface of HUVEC cells that were transfected with the GFP control vector (unfilled curve), and the TNFR1-4-2-Kox1 peptide (red filled curve). The results are shown for all cells which show GFP fluorescence at levels of 10-fold or more above background. As expected, the HUVEC cells transfected with just GFP containing vectors expressed TNFR1 on the cell surface to the same level as untransfected control cells. In contrast, the cells transfected with the TNFR1 specific repressor protein, TNFR1-4-2-Kox1 clearly demonstrate a population of cells (60% of all cells expressing GFP), which do not express TNFR1. These results demonstrate the down-regulation of the TNFR1 protein by a zinc finger repressor protein, specifically targeted to its promoter.

The above experiments show that zinc finger polypeptides engineered against a TNFR1 receptor nucleotide sequence is capable of down-regulating expression of receptor polypeptides in primary cells.

### Example 7

### Specific Up-regulation of Endogenous Erythropoietin In Primary Cells (FACS Analysis)

A. Target DNA Sequences in the Human Erythropoietin Promoter

The 5′ upstream region of the human erythropoietin gene (Genbank Accession No. E15771) from −1 to −1165 is scanned for potential zinc finger binding sites, and a guanine rich region at around −840 is selected as a target site. The region of the human erythropoietin gene promoter from −830 to −860 is shown below, with 9 bp target DNA sequences underlined.

```
  −830 −860
5′ TGTCTGGGGTG GGGGCTGGGTGCGGTGGCTCA 3′
                A B
```

Three finger peptides are selected using the ‘bipartite’ selection protocol as detailed in our PCT publication number WO98/53057, to bind the 9 bp sites (A and B) underlined.

B. Construction of 6-Zinc Finger Peptide to Activate Human Erythropoietin Gene Expression

Having selected 3-finger peptide units to bind the 9 bp A and B sequences above, standard PCR is used to fuse together these 3-finger peptides with the linker peptide—TGSERP-, to generate a 6-finger peptide, called EPOb-a, which recognises the 18 bp target site B-A.

The selected amino acid residues in the helical regions of each zinc finger of EPOb-a are shown below. Residues are numbered relative to the first position in the α-helix (position 1) in each finger (F1-6).

EPOb-a (Linker TGSERP between F3 and F4)

| F1 | F2 | F3 | F4 | F5 | F6 |
|---|---|---|---|---|---|
| −1123456 | −1123456 | −1123456 | −1123456 | −1123456 | −1123456 |
| NSDHLTE | QRSDLSR | RNDHRTK | RSDELTR | RSDHLSE | RKHDRTK |

The 6-zinc finger peptide selected to bind to the EPO promoter is then engineered into a transcriptional activator, which contains three further protein domains. The second domain is the 7 amino acid nuclear localisation sequence (NLS) of the wild-type Simian Virus 40 large-T antigen (Kalderon et al., Cell 39:499-509 (1984), which is fused to the C-terminus of the zinc finger peptide, to direct the activator peptide to the nucleus. Following the NLS, a tetramer of the transactivation domain from the Herpes Simplex Virus (HSV), VP64 is fused to the construct. (VP16, which is the minimal transactivation domain from HSV may also be used).

The fourth domain is the 9E10 region that corresponds to a myc epitope tag, and allows the specific antibody recognition of the expressed zinc finger chimeric peptide in cells, if required. This region is fused to the extreme C-terminus of the peptide. The final, four-domain peptide is called EPOb-a-VP64 and the complete amino acid sequence of this peptide is shown below. The peptide sequence of the 6 zinc fingers is shown in bold.

MAERPYACPVESCDRRFSNSDHLTEHIRIHTGQKPFQCRICMRNFSQRSDLSRHI
RTHTGEKPFACDICGRKFARNDHRTKHTKIHTGSERPYACPVESCDRRFSRSDEL
TRHIRIHTGQKPFQCRICMRNFSRSDHLSEHIRTHTGEKPFACDICGGKFARKHD
RTKHTKIHLRQKDAARNSGPKKKRKVELQLTSDALDDFDLDMLGSDALDDFDLDML
GSDALDDFDLDMLGSDALDDFDLDMLSSQLSQEQKLISEEDL

C. Binding Activity of the EPOb-a-VP64 Peptide

In Vitro Binding Affinity/Specificity

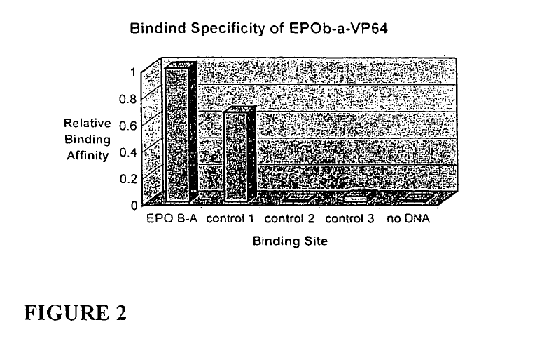

The binding affinity and specificity of the EPOb-a-VP64 peptide is first assayed using the in vitro fluorescence ELISA protocol outlined above (Example 2). The results shown in FIG. 2 show the binding of the 6-zinc finger peptide, EPOb-a-VP64, to its preferred target site (EPO B-A), to three control sites (control 1, 2, 3), and against a no-binding site control (no DNA). The sequences of each binding site are shown below the graph. The control sites 1 and 2 contain mutations in the target DNA sequence (underlined), and control site 3 contains a 3 bp deletion with respect to the target binding site.

This analysis demonstrates that the 6-finger peptide binds tightly and specifically to its selected target site, EPO B-A.

In Vivo Activity (Erythropoietin Expression)

The activity of the EPOb-a-VP64 peptide as a transcriptional activator in vivo, is assayed in the same way as the transcriptional repression activity of the TNFR1-4-2-Kox1 peptide described above.

The EPOb-a-VP64 peptide is cloned into a viral vector to facilitate transduction as described in Example 4 above. Oncoviral particles are purified from helper cells, as described, and the EPOb-a-VP64 peptide-containing vector is used to infect HUVEC cells, as above, or human dermal fibroblast (HDF) cells (Clonetics).

After transfection, cells are maintained for 48 hours, at which point transfection efficiency is assessed on the basis of GFP expression. If transfection efficiency is low (i.e. below about 50%), cells are sorted using FACS analysis using the MoFlo machine (Cytomation). Cells expressing GFP (and therefore, the zinc finger peptide) are collected and maintained further.

Samples of supernatant are taken after 24, 48 and 72 hours to assay for EPO expression, using a commercially available Human Erythropoietin ELISA kit (R&D Systems), according to the manufacturers instructions. First a standard curve is plotted, based on known concentrations of erythropoietin. This curve is then used to calculate the concentration of EPO in the supernatant of the cells expressing the EPOb-a-VP64 peptide.

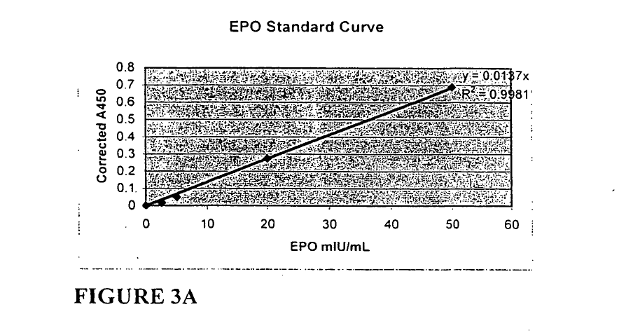

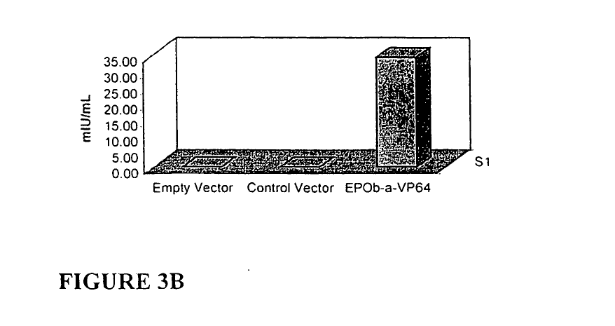

FIG. 3A shows the standard erythropoietin curve obtained from the ELISA kit. The graph of FIG. 3B shows the amount of EPO secreted from primary HDF cells 48 hours after FACS sorting the transfected population. The three samples shown are from transfections with: empty viral vector, expressing just GFP; a control vector expressing a 6-finger-VP64 peptide which is not designed-to bind to the human EPO promoter; the viral vector expressing EPOb-a-VP64. Values shown are in milliunits of EPO per ml (mlU/ml) of media. Instead of, or in addition to the detection of EPO using ELISA, levels of EPO mRNA may be determined using RT-PCR with EPO mRNA-specific primers as described above.

Normal levels of EPO in the plasma and serum of humans range between 3.3 and 16.6 mlU/ml, but average at around 6-7 mlU/ml. The 6-finger peptide, EPOb-a-VP64, is seen to activate the EPO gene to the extent that over 30 mlU/ml of EPO is detected in the media of transfected cells.

The results of this study clearly demonstrate the activation of the endogenous human erythropoietin gene in human primary cells, specifically primary HDF cells.

### REFERENCES

1. Berger, E. a.; Doms, R. W.; Fenyo, E. M.; Korber, B. T.; Littman, D. R.; Moore, J. P.; Sattentau, Q. J.; Schuitemaker, H.; Sodroski, J.; Weiss, R. a., a New Classification for Hiv-1. Nature 391(6664):240 (1998).

2. Bleul, C. C; Farzan, M.; Choe, H.; Parolin, C.; Clark-Lewis, I.; Sodroski, J.; Springer, T. a., the Lymphocyte Chemoattractant Sdf-1 is a Ligand for Lestr/Fusin and Blocks Hiv-1 Entry. Nature 382(6594):829-833 (1996).

3. Bleul, C. C.; Wu, L.; Hoxie, J. a.; Springer, T. a.; Mackay, C. R., the Hiv Coreceptors Cxcr4 and Ccr5 Are Differentially Expressed and Regulated On Human T Lymphocytes. Proc Natl Acad Sci Usa 94(5):1925-1930 (1997).

4. Brelot, a.; Heveker, N.; Pleskoff, O.; Sol, N.; Alizon, M., Role of the First and Third Extracellular Domains of Cxcr-4 in Human Immunodeficiency Virus Coreceptor Activity. J Virol 71(6):4744-4751 (1997).

5. Di Marzio, P.; Tse, J.; Landau, N. R., Chemokine Receptor Regulation and Hiv Type 1 Tropism in Monocyte-Macrophages. Aids Res Hum Retroviruses 14(2):129-138 (1998).

6. D'souza, M. P.; Harden, V. a., Chemokines and Hiv-1 Second Receptors. Nature Medicine 2:1293-1300 (1996).

7. Feng, Y.; Broder, C. C.; Kennedy P. E.; Berger E. a., Hiv-1 Entry Cofactor: Functional C-Dna Cloning of a Seven-Transmembrane, G Protein-Coupled Receptor. Science 272:872-877 (1996).

8. Forster, R.; Kremmer, E.; Schubel, a.; Breitfeld, D.; Kleinschmidt, a.; Nerl, C.; Bernhardt, G.; Lipp, M.; Intracellular and Surface Expression of the Hiv-1 Coreceptor Cxcr4/Fusin On Various Leukocyte Subsets: Rapid Internalization and Recycling Upon Activation. J Immunol 160(3):1522-1531 (1998).

9. Granelli-Pipemo, a.; Moser, B.; Pope, M.; Chen, D.; Wei, Y.; Isdell, F.; O'doherty, U.; Paxton, W.; Koup, R.; Mojsov, S.; Bhardwaj, N.; Clark-Lewis, I.; Baggiolini, M.; Steinman, R. M.; Efficient Interaction of Hiv-1 with Purified Dendritic Cells Via Multiple Chemokine Coreceptors. J Exp Med 184(6):2433-2438 (1996).

10. Gupta, S. K.; Lysko, P. G.; Pillarisetti, K.; Ohlstein, E.; Stadel, J. M.; Chemokine Receptors in Human Endothelial Cells—Functional Expression of Cxcr4 and Its Transcriptional Regulation By Inflammatory Cytokines. J Biol Chem 273(7):4282-4287 (1998).

11. Lavi, E.; Strizki, J. M.; Ulrich, a. M.; Zhang, W.; Fu, L.; Wang, Q.; O'connor, M.; Hoxie, J. a.; Gonzalez-Scarano, F.; Cxcr-4 (Fusin), a Co-Receptor for the Type 1 Human Immunodeficiency Virus (Hiv-1), is Expressed in the Human Brain in a Variety of Cell Types, Including Microglia and Neurons. Am J Pathol 151(4): 1035-1042 (1997).

12. Lu, Z. H.; Berson, J. F.; Chen, Y. H.; Turner, J. D.; Zhang, T. Y.; Sharron, M.; Jenks, M. H.; Wang, Z. X.; Kim, J.; Rucker, J.; Hoxie, J. a.; Peiper, S. C.; Doms, R. W., Evolution of Hiv-1 Coreceptor Usage Through Interactions with Distinct Ccr5 and Cxcr4 Domains. Proc Natl Acad Sci Usa 94(12):6426-6431 (1997).

13. Oberlin, E.; Amara, a.; Bachelerie, F.; Bessia, C.; Virelizier, J.-L.; Arenzana-Seisdedos, F.; Schwartz, O.; Heard, J.-M.; Clark-Lewis, I.; Legler, D. F.; Loetscher, M.; Baggiolini, M.; Moser, B., the Cxc Chemokine Sdf-1 is the Ligand for Lestr/Fusin and Prevents Infection By T-Cell-Line-Adapted Hiv-1. Nature 382(6594):833-835 (1996).

14. Signoret, N.; Oldridge, J.; Pelchen-Matthews, a.; Klasse, P. J.; Tran, T.; Brass, L. F.; Rosenkilde, M. M.; Schwartz, T. W.; Holmes, W.; Dallas, W.; Luther, M. a.; Wells, T. N. C.; Hoxie, J. a.; Marsh, M., Phorbol Esters and Sdf-1 Induce Rapid Endocytosis and Down Modulation of the Chemokine Receptor Cxcr4. J Cell Biol 139(3):651-664 (1997).

15. Volin, M. V.; Joseph, E.; Shockley, M. S.; Davies, P. F., Chemokine Receptor Cxcr4 Expression in Endothelium. Biochem Biophys Res Commun 242(1):46-53 (1998).

16. Yi, Y. J.; Rana, S.; Turner, J. D.; Gaddis, N.; Collman, R. G., Cxcr-4 is Expressed By Primary Macrophages and Supports Ccr5-Independent Infection By Dual-Tropic But Not T-Tropic Isolates of Human Immunodeficiency Virus Type 1. J Virol 72(1):772-777 (1998).

17. Chan, F. K., Siegel, R. M., Lenardo, M. J., Signaling by the TNF Receptor Superfamily and T Cell Homeostasis. Immunity 13: 419-422 (2000).

18. Suvannavejh, G. C., Lee, H. O., Padilla, J., Dal Canto, M. C., Barret, T. A., Miller, S. D., Divergent roles for p55 and p75 tumour necrosis factor receptors in the pathogenesis of MOG(35-55)-induced experimental autoimmune encephalomyelitis. Cell. Immunology 205: 24-33 (2000).

19. Immunology. Eds Roitt, I., Brostoff, J., Male, D. Mosby, London, 4^{th} edition (1996).

20. Maini, R. N., Taylor, P. C., Paleolog, E., Charles, P., Ballara, S., Brennan, F. M., Feldmann, M., Anti-tumour necrosis factor specific antibody (infliximab) treatment provides insights into the pathophysiology of rheumatoid arthritis. Ann. Rheum. Disease 58:156-160 (1999).

21. Garrison, L., McDonnell, N. D., Etanercept: therapeutic use in patients with rheumatoid arthritis. Ann. Rheum. Disease58:165-169 (1999).

22. Bachmaier, K., Pummerer, C., Kozieradzki, I., Pfeffer, K., Mak, T. W., Neu., N., Penninger, J. M. Low-molecular-weight tumour necrosis factor receptor p55 controls induction of autoimmune heart disease. Circulation 95: 655-661 (1997).

23. Li, Y. Y., Feng, Y. Q., Kadokami, T., Mctiernan, C. F., Draviam, R., Watkins, S. C., Feldman, A. M., Myocardial extracellular matrix remodeling in transgenic mice overexpressing tumor necrosis factor α can be modulated by anti-tumor necrosis factor cc therapy. Proc. Natl. Acad. Sci. USA 97: 12746-12751 (2000).

24. Kollias, G., Douni, E., Kassiotis, G., Kontoyiannis, D., On the role of tumour necrosis factor and receptors in models of multi-organ failure, rheumatoid arthritis, multiple sclerosis and inflammatory bowel disease. Immunol. Rev. 169: 175-194 (1999).

25. Kemper, O., Wallach, D., Cloning and partial characterization of the promoter for the human p55 tumor necrosis factor (TNF) receptor. Gene 134: 209-216 (1993).

26. Luscher B, Mitchell P J, Williams T, Tjian R. Regulation of transcriptional factor AP-2 by the morphogen retinoic acid and by second messengers. Genes Dev. 3:1507-1517 (1989).

Each of the applications and patents mentioned above, and each document cited or referenced in each of the foregoing applications and patents, including during the prosecution of each of the foregoing applications and patents (“application cited documents”) and any manufacturer's instructions or catalogues for any products cited or mentioned in each of the foregoing applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference. In particular, we hereby incorporate by reference International Patent Application Numbers PCT/GB00/02080, PCT/GB00/02071, PCT/GB00/03765, United Kingdom Patent Application Numbers GB0001582.6, GB0001578.4, and GB9912635.1 as well as U.S.09/478,513.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method of regulating expression of a nucleic acid sequence in a primary cell, the method comprising providing a nucleic acid binding polypeptide capable of binding to the nucleic acid sequence, and contacting the nucleic acid binding polypeptide with the nucleic acid sequence in the primary cell to regulate its expression.

2. A nucleic acid binding polypeptide capable of binding to and regulating the expression of a nucleic acid sequence in a primary cell.

3. A method according to claim 1 or a nucleic acid binding polypeptide according to claim 2, in which the nucleic acid sequence comprises an endogenous cellular gene.

4. A method or polypeptide according to claim 3, in which the nucleic acid binding polypeptide is capable of binding to a promoter or other control sequence of the endogenous gene.

5. A method or polypeptide according to any preceding claim, in which the nucleic acid binding polypeptide is provided by expression from an expression vector which is introduced into the primary cell or an ancestor of the primary cell.

6. A method or polypeptide according to any preceding claim, in which the nucleic acid binding polypeptide comprises a zinc finger polypeptide.

7. A method or polypeptide according to any preceding claim, in which the primary cell comprises an untransformed cell.

8. A method or polypeptide according to any preceding claim, in which the nucleic acid binding polypeptide comprises a transcriptional repression domain selected from the group consisting of: a KRAB domain, an engrailed domain and a snag domain.

9. A method or polypeptide according to any of claims 1 to 7, in which the nucleic acid binding polypeptide comprises a transcriptional activation domain selected from the group consisting of: VP16, VP64, transactivation domain 1 of the p65 subunit (RelA) of nuclear factor-κB, transactivation domain 2 of the p65 subunit (RelA) of nuclear factor-κB, and the activation domain of CTCF.

10. A method or polypeptide according to any preceding claim, in which the primary cell is introduced into an organism.

11. A method or polypeptide according to any preceding claim, in which the nucleic acid sequence is capable of encoding erythropoietin (EPO) or TNF receptor 1 (TNFR1).

12. A primary cell comprising an exogenous nucleic acid binding polypeptide, the nucleic acid binding polypeptide capable of regulating the expression of a nucleic acid sequence of the primary cell.

13. A pharmaceutical composition comprising a polypeptide according to any of claims 1 to 11 or a primary cell according to claim 12, together with a pharmaceutically acceptable carrier or diluent.

14. A method of treating or preventing a disease in a patient, the method comprising the steps of:
(a) providing a primary cell;
(b) introducing a nucleic acid binding polypeptide into the primary cell, in which the nucleic acid binding polypeptide binds to and regulates a nucleic acid sequence responsible for or associated with the disease; and
(c) introducing the primary cell into the patient.

15. A method according to claim 14, in which the primary cell is provided from the patient to be treated.

16. A method of expressing a protein in a primary cell, the method comprising the steps of:
(a) providing a primary cell comprising a nucleic acid sequence encoding a protein;
(b) introducing a nucleic acid binding polypeptide into the primary cell, in which the nucleic acid binding polypeptide binds to and promotes the expression of the protein from the nucleic acid sequence.

17. A method according to claim 16, in which the primary cell is of a cell type which does not normally express the protein.

18. A method of expressing an exogenous nucleic acid binding polypeptide in a primary cell, the method comprising the steps of:
(a) providing a nucleic acid sequence encoding a nucleic acid binding polypeptide operatively linked to a control sequence;
(b) introducing the nucleic acid sequence into the primary cell, or an ancestor of the primary cell; and
(c) allowing the nucleic acid binding polypeptide to be expressed from the nucleic acid sequence within the primary cell.
